(19) United States
(12) Patent Application Publication
(10) Pub. No.: US 2017/0321196 A1
(43) Pub. Date: Nov. 9, 2017

(54) ANALYTICAL AND DIAGNOSTIC METHODS UTILIZING SHIGELLA FLEXNERI APYRASE

(71) Applicant: ApiRays AB, Solna (SE)

(72) Inventors: Pavankumar Asalapuram, Huddinge (SE); Aman Russom, Solna (SE)

(21) Appl. No.: 15/524,160

(22) PCT Filed: Nov. 6, 2015
(86) PCT No.: PCT/EP2015/075924

§ 371 Date: May 3, 2017

(30) Foreign Application Priority Data
Nov. 7, 2014 (SE) 1451332-9

(51) Int. Cl. C12N 9/14; C12Q 1/00; C12Q 1/34

(52) U.S. Cl. CPC C12N 9/14; C12Q 1/34; C12Q 1/008; G01N 2333/90

(57) ABSTRACT

A method, comprising the steps of providing a sample containing contaminating nucleoside diphosphates and/or nucleoside triphosphates, such as ATP and/or ATP analogues including deoxyribonucleoside triphosphates; reducing the amount of the contaminating nucleoside diphosphates and/or nucleoside triphosphates in the sample with an apyrase enzyme, wherein said apyrase enzyme is a Shigella flexneri apyrase; and performing an analysis of the sample, wherein said analysis comprises an assay that would have been affected by the contaminating nucleoside diphosphates and/or nucleoside triphosphates had they not been reduced in the reduction step.

### TECHNICAL FIELD

The present invention relates to analytical and diagnostic methods where contaminating nucleotides are an issue. In particular, the present invention relates to determining or quantifying the amount of ATP present in a sample that may contain contaminating nucleotides, as well as reagents for use in such methods and production of such reagents.

### BACKGROUND

Adenosine triphosphate (ATP) is a molecule present in all living cells. Since the concentration of ATP is fairly constant in the cell, measurement of ATP content in a sample can be used as a proxy to determine the number of viable cells. Sensitive bioluminescent assays for measuring ATP based on luciferase/luciferin are known, see e.g. U.S. Pat. No. 3,745,090. Luciferase (e.g. from firefly) is a euglobulin protein that catalyses the oxidative decarboxylation of luciferin using ATP and molecular oxygen to yield oxyluciferin, a highly unstable, single-stage excited compound that emits light upon relaxation to its ground state. This reaction emits light proportional to ATP concentration in the reaction mixture, and by measuring the intensity of the emitted light it is possible to continuously monitor the concentration of ATP present.

In many cases, samples to be analyzed for ATP content contain contaminating ATP either extracellularly or present in a contaminating type of cells. For instance, if the number of bacteria is to be quantitated in any clinical or biological sample, any ATP contained in host cells present in the sample will interfere with the measurement. In many applications, the contaminating cells may be selectively lysed and the ATP released, resulting in that all the contaminating ATP is in extracellular form, see e. g. U.S. Pat. No. 4,303,752 or US20110076706. In other instances, ATP analogues different from ATP may be present in a sample and interfere with ATP measurement.

The contaminating extracellular ATP, otherwise unwanted ATP or ATP analogues can be reduced or eliminated by hydrolyzing them with an enzyme called apyrase (ATP-diphosphohydrolase, E-type ATPase, ATPDase, NTDase EC 3.6.1.5). Apyrase is frequently used in methods for determining bacterial ATP in the presence of mammalian cells, where the mammalian cells are first selectively lysed and apyrase used to degrade extracellular ATP leaving the bacterial ATP unaffected. After completion of the reaction, the apyrase can be inactivated and the intracellular ATP of bacterial cells is released to measure bacterial ATP by the addition of luciferin/luciferase. Light emission is measured before and after the addition of a known amount of ATP standard, as internal control. The bacterial ATP (in moles) is calculated by multiplying the ratio of the light before and after adding the ATP standard with the amount of added standard. Typically bacterial cells contain around 1 attomole of ATP per cell, making it possible to estimate the number of bacterial cells from the amount of ATP detected. It is an object of the present invention to provide improvements for such analytical and diagnostic methods.

In this context, the most commonly used apyrase is Solanum tuberosum apyrase (STA; commonly known as potato apyrase, SEQ ID NO: 9). STA exists in several isoforms and each isoform differs in ATP-degradation activity. However, the efficiency of STA in the above methods is limited by the accumulation of ADP and uncharacterized ATP-analogues in the degradation reaction when using STA. Accumulation of such contaminants inhibit the ATP degradation capability allowing some of the contaminating ATP to remain intact, which in turn limits the sensitivity of the ATP determination assays and also increases the background signal. For a discussion on the limitations of STA, see W0199402816.

Further, most embodiments of the DNA sequencing method pyrosequencing (see e.g. international patent applications PCT/GB1997/002631 and PCT/GB1997/003518, or US patent applications US2013/0045876 and US2013/0189717) also rely on quantitation of ATP, usually by the luciferase/luciferin assay. At the end of every sequencing cycle, all the unincorporated nucleotides and excess ATP must be eliminated by e.g. apyrase. As detailed above, the apyrases presently used in DNA sequencing applications have problems in achieving complete degradation due to the quality of enzyme (contamination of NDP kinase), substrate specificity and batch-to-batch variations that not only results in drop off and non-linear peaks but also affects DNA sequencing of long strands. It is an object of the present invention to provide better elimination of ATP, its analogues as well as other di- or triphosphate nucleotides to e.g. improve DNA sequencing.

Definitions

The following terms and words have the meanings as defined below in the context of the present disclosure.

The words comprise or comprising is to be interpreted in a non-limiting fashion, such as to include or to contain; have as a component.

Shigella flexneri apyrase (SFA) is defined as apyrase derived from Shigella flexneri, at any suitable degree of purity. The SFA may be produced by non-recombinant means or by recombinant DNA technology. Recombinant Shigella flexneri apyrase is abbreviated rSFA herein. The native SFA has the sequence according to SEQ ID NO: 10. The SFA according to the definition has apyrase activity substantially similar to the rSFA according to SEQ ID NO. 4 or SFA according to SEQ ID NO: 10. Apyrase activity refers to capacity to catalyse hydrolysis of nucleoside triphosphates to nucleoside diphosphates, and hydrolysis of nucleoside diphosphates to nucleoside monophosphates.

Sequence identity expressed in percentage is defined as the value determined by comparing two optimally aligned sequences over a comparison window, wherein a portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Unless indicated otherwise, the comparison window is the entire length of the sequence being referred to. In this context, optimal alignment is the alignment produced by the BLASTP algorithm as implemented online by the US National Center for Biotechnology Information (see The NCBI Handbook [Internet], Chapter 16), with the following input parameters: Word length=3, Matrix=BLOSUM62, Gap cost=11, Gap extension cost=1.

ATP analogues refer to compounds with structural and functional similarity to ATP which compete with ATP for binding to enzymes that specifically interact with ATP. In the present context, the term only applies to compounds that are substrates for SFA as defined above.

Preferably, the term refers to compounds that can substitute ATP as substrate for luciferases, such as firefly luciferase. In preferable embodiments, the term includes nucleoside diphosphates and nucleoside triphosphates. In more preferable embodiments, the term refers to adenosine diphosphate, deoxyadenosine alpha-thio triphosphate, adenosine tetra-phosphate, deoxyadenosine triphosphate, deoxyadenosine diphosphate, guanosine triphosphate, guanosine diphosphate, deoxyguanosine triphosphate, deoxyguanosine diphosphate, thymidine triphosphate, deoxythymidine triphosphate, thymidine diphosphate, deoxythymidine triphosphate, cytidine diphosphate, deoxycytidine triphosphate, cytidine triphosphate, deoxycytidine diphosphate, uridine diphosphate and uridine triphosphate. In most preferable embodiments, ATP analogues refers to: adenosine diphosphate, deoxyadenosine alpha-thio triphosphate, adenosine tetra-phosphate, deoxyadenosine triphosphate, deoxyadenosine diphosphate, guanosine triphosphate, guanosine diphosphate, deoxyguanosine triphosphate and deoxyguanosine diphosphate.

### SUMMARY OF THE INVENTION

Briefly, the present invention relates to the following items. The subject matter disclosed in the items below should be regarded disclosed in the same manner as if the subject matter were disclosed in patent claims.
1. A method, comprising the steps of
   a. providing a sample containing contaminating nucleoside diphosphates and/or nucleoside triphosphates, such as ATP and/or ATP analogues including deoxyribonucleoside triphosphates;
   b. reducing the amount of the contaminating nucleoside diphosphates and/or nucleoside triphosphates in the sample with an apyrase enzyme, wherein said apyrase enzyme is a Shigella flexneri apyrase; and
   c. performing an analysis of the sample, wherein said analysis comprises an assay that would have been affected by the contaminating nucleoside diphosphates and/or nucleoside triphosphates had they not been reduced in step b.
2. The method according to item 1, being a method for determining the amount of ATP in a sample, comprising the steps of:
   a. reducing the amount of contaminating ATP and/or ATP analogues in the sample by degradation with an apyrase enzyme;
   b. making the ATP to be determined available for determination; and
   c. determining the amount of ATP to be determined in the sample, wherein the apyrase enzyme is a Shigella flexneri apyrase.
3. The method according to item 2, wherein the method is for determining the amount of ATP present in a first population of cells in a sample, comprising the steps of:
   a. reducing the amount of contaminating ATP and/or ATP analogues in the sample by degradation with an apyrase enzyme;
   b. liberating the ATP to be determined from the first population of cells; and
   c. determining the amount of liberated ATP; wherein the apyrase enzyme in step (a) is a Shigella flexneri apyrase.
4. The method according to item 3, wherein the liberation step (b) involves lysis of the first population of cells.
5. The method according to any of items 3-4, wherein the first population of cells comprises bacterial cells.
6. The method according to any of items 1-5, wherein the sample is a biological sample from an animal.
7. The method according to item 6, wherein the sample is a biological sample from a human.
8. The method according to any of items 1-7, wherein the sample is a blood sample, a plasma sample, a serum sample, a urine sample, a faecal sample, or a swab from a patient.
9. The method according to any of items 2-8, wherein at least a fraction of the contaminating ATP is present in a second population of cells, and the reduction step (a) is preceded by a step of selective liberation of ATP from the second population of cells.
10. The method according to item 9, wherein the second population of cells are host cells from an animal from which the sample is derived.
11. The method according to any of items 1-10, where the method comprises the step of adding an apyrase inhibitor after the reduction step.
12. The method according to item 11, wherein the apyrase inhibitor comprises ortho-vanadate or Mg²⁺, preferably ortho-vanadate.
13. The method according to item 12, wherein ortho-vanadate is present in the sample at a concentration of at least 0.1 mM, preferably 0.2-25 mM, more preferably 0.5-20 mM, or Mg²⁺ is present in the sample at a concentration of at least 25 mM, preferably 30-200 mM, more preferably 30-150 mM.
14. The method according to any of items 1-5 or 11-13, wherein the sample is a cell culture medium sample, food sample, a beverage sample, a pharmaceutical sample, a sewage sample, an environmental sample such as a swab from a surface, or a drinking water sample.
15. The method according to any of items 3-14, comprising step (d) of calculating the number or concentration of viable cells of the first population present in the sample from the amount of ATP determined in step (c).
16. The method according to item 1, wherein the method is a sequencing-by-synthesis procedure, the contaminating nucleotides comprise excess dNTPs or analogues thereof present after a completed sequencing cycle, and the analysis performed on the sample is a sequence readout.
17. The method according to item 1 or 16, wherein the method is for doing pyrosequencing and comprises the steps of:
   a. performing a pyrosequencing reaction comprising addition of a nucleoside triphosphate;
   b. converting the pyrophosphate released in step (a) into ATP via an enzymatic reaction;
   c. determining the amount of ATP formed in step (b);
   d. degrading unincorporated nucleoside triphosphate from step (a) and ATP formed in step (b) with an apyrase enzyme;
   e. repeating the steps a-d at least once;
   wherein the apyrase enzyme is a Shigella flexneri apyrase.
18. The method according to any of items 2-15, wherein the determination of the amount of ATP is performed with a bioluminescent assay.
19. The method according to item 18, wherein the determination of the amount of ATP is performed with bioluminescent assay utilizing luciferin and luciferase.
20. The method according to item 19, wherein luciferase is provided in a composition according to any of items 38-39.
21. The method according to any of the preceding items, wherein the Shigella flexneri apyrase comprises an amino-acid sequence with at least 80%, more preferably at least 85%, even more preferably at least 90%, yet more preferably at least 95%, still more preferably at least 97%, most preferably 100% sequence identity to SEQ ID NO: 10.
22. The method according to any of the preceding items, wherein the Shigella flexneri apyrase comprises an amino-acid sequence according to SEQ ID NO: 4.
23. The method according to any of the preceding items, wherein Shigella flexneri apyrase is provided in a composition according to any of items 36-37.
24. A Shigella flexneri apyrase comprising an amino-acid sequence with at least 95%, preferably at least 96%, more preferably at least 97%, yet more preferably at least 98%, still more preferably at least 99% sequence identity to SEQ ID NO: 4 and having apyrase activity.
25. The Shigella flexneri apyrase according to item 24, with an amino-acid sequence according to SEQ ID NO: 4.
26. A polynucleotide encoding an apyrase according to item 24 or 25.
27. A vector comprising a polynucleotide according to item 26 operably linked to a promoter capable of inducing expression of the polynucleotide.
28. A host cell comprising a polynucleotide according to item 27 operably linked to a promoter capable of inducing expression of the polynucleotide or a vector according to item 21.
29. A method of producing a recombinant apyrase according to item 24 or 25, comprising the steps of:
   a. culturing host cells according to item 28 in conditions inducing the expression of the polynucleotide; and
   b. recovering the produced recombinant apyrase from the culture.
30. A use of a Shigella flexneri apyrase for degrading contaminating nucleoside triphosphates or nucleoside disphosphates in an analytical method.
31. The use according to item 30, wherein the contaminating nucleosides comprise contaminating ATP and/or ATP analogues, and the analytical method comprises an assay for measuring ATP.
32. The use according to item 31, wherein the assay for measuring ATP is a bioluminescent assay.
33. A use of a Shigella flexneri apyrase in a sequencing by synthesis assay, preferably a pyrosequencing assay.
34. The use according to any of items 30-33, wherein the apyrase comprises an amino-acid sequence with at least 80%, more preferably at least 85%, even more preferably at least 90%, yet more preferably at least 95%, still more preferably at least 97%, most preferably 100% sequence identity to SEQ ID NO: 10.
35. The use according to any of items 30-34, wherein the apyrase is according to any of items 24-25.
36. A composition comprising a Shigella flexneri-apyrase in a buffer having a pH in the range of 6-9, preferably 7-8, and an ionic strength of at least 300 mM.
37. The composition according to item 36, wherein the ionic strength is 300-1000 mM, preferably 400-800 mM, more preferably 500-800 mM.
38. A composition comprising a luciferase and an apyrase inhibitor comprising ortho-vanadate.
39. The composition according to item 38, wherein the concentration of ortho-vanadate is at least 0.2 mM, preferably at least 1 mM, more preferably 1-3000 mM, most preferably 2-200 mM.

### BRIEF DESCRIPTION OF THE DRAWINGS

FIG. 1. Colorimetric evaluation of recombinant Shigella flexineri apyrase (rSFA). The overexpressed rSFA was tested colorimetrically using ammonium molybdate and ferrous ammonium sulphate to check the localization, quality and efficiency. Sub-cellular fractions of E. coli BL21-A1 containing apyrase enzyme were tested and the optical densities (O.D.) of these fractions were measured in colorimeter to make a qualitative and/or quantitative correlation. Here, the inventors have made a qualitative comparison to localize the expression of apyrase enzyme among certain sub-cellular fractions of the developed clone and show its high activity through colorimetric analysis.

FIG. 2. Sub-cellular localization of rSFA enzyme expression. Sub-cellular fractions of the E. coli BL21-A1 expression vector bearing the Shigella flexineri apyrase (SFA) gene with 6-histidines in pBL plasmid were resolved on 10% SDS-PAGE to check the expression of the enzyme. Expression of rSFA was found 25 kDa in fractions of induced soluble samples, which was used to purify the apyrase enzyme. The non-induced and induced samples of purified fractions and the whole cell lysates, respectively. This experiment showed that the enzyme is localized in the soluble fractions of the expression host.

FIG. 3. Sequence analysis of rSFA

The transformed clone of rSFA bearing apyrase gene with 6x Histidines was sequenced by Sanger's method. The cloned fragment contained 780 homologous bases (FIG. 3A) of 1134 nucleotides of virulent SFA. FIG. 3B illustrates the similarity between the native SFA reference sequence (SEQ ID NO: 10, termed “SFA”) and rSFA (SEQ ID NO: 4, termed “rSFA”. The sequences differ only at the N- and the C-termini.

FIG. 4. Comparison between the activities of recombinant Shigella flexineri apyrase (rSFA) and potato apyrase (STA) activities

The enzymes, rSFA and STA diluted in 10× in Tris-EDTA Buffer were tested to compare their ATP degradation efficiencies. As can be seen in the figure, rSFA was very efficient in eliminating the ATP, while the commercial STA found to be slower. Even after 60 min of reaction, STA could not eliminate ATP completely, while rSFA eliminated ATP in ^{˜}15 min with a rate constant of 3.87 min⁻¹. Light was measured in the fully automatic 1251 Luminometer (LKB-Wallac, Turku, Finland).

FIG. 5. Effect of rSFA and STA on ATP and light emission

The effect of the addition of recombinant Shigella flexineri apyrase (rSFA; diamonds), potato apyrase (STA; squares) and ATP on the light emission from the firefly luciferase reaction. The reaction mixture consisted of 0.2 mL ATP Reagent SL and 0.8 mL Tris-EDTA Buffer (BioThema AB, Sweden). The 1^{st}, 3^{rd} and 4^{th} arrows denote ATP addition and the 2^{nd} arrow apyrase addition. The light was measured every 15 sec except at the additions. Ten μL of 10 μmol/L ATP was added along with 10 μL apyrase. Light was measured in the fully automatic 1251 Luminometer (LKB-Wallac, Turku, Finland).

FIG. 6. Elimination of extracellular ATP in serum

Comparison of ATP elimination with rSFA and STA. In luminescence analysis with 100% crude serum (FIG. 6A), and 10% diluted serum samples (FIG. 6B), rSFA shows very good ATP elimination activity compared to that of STA.

FIG. 7. Comparison of the activity of rSFA with different commercial STA sources

Comparison of three different commercial products of potato apyrase termed STA, A6535 and A6237, to rSFA in buffer. rSFA exhibits superior results.

FIG. 8. Comparison of the activity of rSFA with different commercial STA sources in 10% serum

Comparison of commercial products of potato apyrase like STA, A6535 and A6237 to rSFA in 10% serum. rSFA exhibits superior results.

FIG. 9. Comparison of the activity of rSFA to commercial STA, in urine

Comparison of commercial STA to rSFA in urine, rSFA exhibits superior results. Though both the enzymes showed similar initial activities, rate of action of STA was hindered after 3 min, whereas rSFA continued with ATP-elimination. Even after 4 subsequent injections of 10 μL 10 μmol/L ATP, the rSFA showed a complete ATP elimination activity each time in less than 3 min but STA could not digest the ATP even after 20 min.

FIG. 10. Effect of ATP-analogues against STA and rSFA in urine

Elimination of ADP at 10 μL 10 μmol/L and a mixture of ATP and ADP at 10 μL 10 μmol/L were tested in urine samples. The activity of STA was decreased after either by the addition of ADP or in the mixture of ATP and ADP, whereas the activity of rSFA was not affected.

FIG. 11. Optimization of buffer system to improve the activity of rSFA

Equal concentration of rSFA was stored in Buffer 1, Buffer 2 and Buffer 3 for about 10 months and the ATP-elimination assays were performed. The activity of the enzyme was checked intermittently and found that the enzyme used with Buffer 3 showed very good activity compared with other two buffers

FIG. 12. Development of inhibitors for rSFA

Among the different inhibitors tested, 100 mM of MgSO₄ and 1 mM of ortho-vanadate in the form of Na₃VO₄ showed inhibitory action against rSFA. The ortho-vanadate at 1 mM did not affect luciferase at all and at the same time, it completely inhibited the activity of rSFA. Magnesium was also able to completely inhibit rSFA at 50 mM, but it also decreased the activity of luciferase.

FIG. 13A-E. Activity of rSFA on ATP, dATP, dTTP, dGTP and dCTP

To compare the rSFA activity on nucleotides to STA, samples of ATP, dATP, dTTP, dGTP and dCTP were incubated with the enzyme for 5 min or 35 min and the products detected using high performance liquid chromatography (HPLC). In all instances rSFA resulted in superior elimination of the nucleotide triphosphates and in particular effectively eliminated nucleotide diphophates, which the STA eliminated rather ineffectively.

### DETAILED DESCRIPTION

The inventors have surprisingly found that Shigella flexneri apyrase (SFA; known as such from WO1994/012211) is more effective in eliminating contaminating or otherwise unwanted nucleoside triphosphates and nucleoside diphosphates, in particular ATP and/or other ATP-analogues, than the commonly used Solanum tuberosum apyrase (STA; potato apyrase).

Table 1 shows a comparison between recombinant Shigella flexneri apyrase (rSFA) and Solanum tuberosum apyrase (STA; potato apyrase). The amount of STA used was slightly higher in terms of units, and after one minute, the amount of ATP remaining was lower in the

STA samples compared to rSFA by nearly a factor of 2. However, at the time point 17 minutes, concentration of ATP in the rSFA treated samples was about 20 times lower compared to STA-treated samples. At 91 minutes the difference was yet more pronounced, the rSFA treated samples having about 45-fold lower concentration than STA-treated samples. It is noteworthy that even after 91 minutes of incubation, the amount of residual ATP was higher in the STA samples compared to the rSFA-samples at 17 minutes.

Without being bound by theory it may be assumed that the greater ability to degrade ATP and its analogues results from one or more of: higher substrate affinity, higher enzyme stability under the assay conditions, lesser inhibition from the reaction end-products. In all ATP-based reactions, presence or accumulation of contaminants or byproducts like ADP, AMP, ATP, dNTP, dNDP, XMP, etc. may interfere with the assay.

Commercial apyrase preparations often contain a mixture of isoenzymes with different relative affinities for ATP, ADP, xMP like tetra- or penta-phosphates, etc. Moreover, the apyrase enzyme activity of different commercial suppliers varies with regard to initial activity and different lots of the same product may show substantially variable ATP-hydrolyzing activities. These variations may be significant obstacles for the development of a robust and sensitive analytical method for analyzing various biological samples.

In this regard, it can be concluded that SFA is much more effective in eliminating traces of above said contaminating ATP and its analogues (or other nucleotides) in a sample that generally hinder the activity and sensitivity of STA. This translates in practical terms that assays for determining the amount of ATP in a sample, where there is or may be contaminating ATP present can achieve greater sensitivity due to better elimination of most of the ATP-analogues, when SFA is used as an apyrase (see Examples 6, 7, 8, and 9). Additionally, in certain circumstances the use of SFA provides for a faster assay by sufficiently eliminating the contaminating ATP in a shorter time. The better elimination of dNTPs (Example 11) also provides for improved sequencing-by-synthesis methods using SFA.

The inventors have also shown that the SFA is not only stable in the reaction conditions but also tolerates freeze-thaw-cycles and storage in solution at refrigerator temperatures, even in crude form, without the addition of stabilizers. The fact that enzyme remains to be active even in high concentrations of EDTA indicates that it does not require any divalent metal ions for its activity, unlike STA. The SFA exhibits very good activity between pH 7.0-7.5, while it remains active between pH 5.0 and 9.5. Therefore the invention provides an opportunity to apply SFA on different biological samples and analytical experiments with a wide range of pH-values. The stability presents a substantial practical advantage, in particular for field laboratories with limited equipment where ATP measurements are performed at times.

Thus, in a first aspect, the present invention relates to a method for reducing the amount of contaminating nucleoside diphosphates and/or nucleoside triphosphates, comprising the steps of
a. providing a sample containing contaminating nucleoside diphosphates and/or nucleoside triphosphates, such as ATP and/or ATP analogues;
b. reducing the amount of the contaminating nucleoside diphosphates and/or nucleoside triphosphates in the sample with an apyrase enzyme, wherein said apyrase enzyme is a Shigella flexneri apyrase; and
c. optionally, performing an analysis of the sample, wherein said analysis comprises an assay that would have been affected by the contaminating nucleoside diphosphates and/or nucleoside triphosphates had they not been reduced in step b.

The assay may involve be determination of the amount or concentration of ATP in the sample. By “would have been affected” is meant that the assay is of such character that contaminating nucleoside diphosphates and triphosphates may potentially have an interfering effect. The recitation is not intended to imply that the reduction in step (b) necessarily results in complete elimination of any and all interference. Rather, even less than complete reduction, including partial reduction, substantial reduction, near complete reduction of interference is encompassed.

The contaminating nucleoside triphosphates may comprise deoxyribonucleoside triphosphates (dATP, dTTP, dGTP, dCTP or analogues thereof), and the analysis may be a sequencing-by-synthesis-assay, such as a pyrosequencing reaction.

By “reducing” is meant that the amount is significantly reduced for practical purposes such that the contaminating nucleoside triphosphates or nucleoside diphosphates do not jeopardize the accuracy of the determination in subsequent steps. Preferably, the reduction results in substantial elimination of the contaminating nucleotide to a level of less than 0.01% of the original amount, more preferably to less than 0.001% of the original amount, most preferably to less than 0.0001% of the original amount.

Methods for Determining ATP in a Sample

The method of the first aspect may be a method for determining the amount of ATP in a sample, comprising the steps of:
a. reducing the amount of contaminating ATP and/or ATP analogues and its analogues in the sample by degradation with an apyrase enzyme;
b. making the ATP to be determined available for determination; and
c. determining the amount of ATP to be measured in the sample, wherein the apyrase enzyme is a Shigella flexneri apyrase.

By “reducing” in the context of ATP determination meant that the amount is significantly reduced for practical purposes such that the contaminating ATP or ATP analogue does not jeopardize the accuracy of the determination in subsequent steps. Preferably, the reduction results in substantial elimination of the contaminating ATP or ATP analogue to a level of less than 0.01% of the original amount, more preferably to less than 0.001% of the original amount, most preferably to less than 0.0001% of the original amount.

The step (b) of making the ATP to be determined available for determination may involve lysis of cells in which the ATP to be determined may be contained, e.g. by heat, denaturing agents, detergents or a combination thereof. The manner of lysis does not matter, as long as the manner used is compatible with the technique used in the determination in step (c).

Alternatively, the ATP may be made available by synthesizing it in the sample, e.g. by way of enzymatic conversion of a different chemical species to ATP.

Methods for Determining the Amount of ATP Present in Cells

The method of the first aspect may be a method for determining the amount of ATP present in first population of cells in a sample, comprising the steps of:
(a) reducing the amount of contaminating ATP and/or ATP analogues in the sample by degradation with an apyrase enzyme;
(b) liberating the ATP to be determined from the first population of cells; and
(c) determining the amount of liberated ATP;
   wherein the apyrase enzyme in step (a) is a Shigella flexneri apyrase.

By “liberating” is meant that the ATP is made accessible for the determination in step (c).

The liberation step (b) may involve lysis of the first population of cells, e.g. by heat, denaturing agents, detergents or a combination thereof. The manner of lysis does not matter, as long as the manner used is compatible with the technique used in the determination in step (c).

The first population of cells may comprise bacterial cells. For instance, it may be of interest to determine the number of bacteria present in a clinical sample. Thus, the sample may a biological sample from an animal, such as a human, a canine, a feline, a bovine, an avian or the like. Preferably, the sample is from a human.

The biological sample may for example be a blood sample, a plasma sample, a serum sample, a urine sample or a fecal sample. The sample may be in the form of a swab from a subject, e.g. from skin or a mucous membrane.

The method of the first aspect may comprise the step of adding an apyrase inhibitor after the reduction step. The apyrase inhibitor added may comprise ortho-vanadate. In the context of the present invention, ortho-vanadate is advantageous since it is able to inhibit Shigella flexneri-apyrase at a concentration which does not inhibit luciferase. Ortho-vanadate as apyrase inhibitor may be present at a concentration of at least 0.1 mM, preferably 0.2-25 mM, more preferably 0.5-20 mM, most preferably about 1 mM in the sample, Alternatively, magnesium can be used as an apyrase inhibitor. Mg²⁺ may be present at a concentration of at least 25 mM, preferably 30-200 mM, more preferably 30-150 mM in the sample.

In the present methods, it may be desirable to inhibit apyrase activity after the contaminating nucleotides have been eliminated, so that the subsequent analysis of the sample is not detrimentally affected by the apyrase. For instance, the apyrase may be inhibited before the liberation of ATP from the first population of cells discussed above, so that the apyrase does not interfere with the determination of the liberated ATP by eliminating some of it.

At least a fraction of the contaminating ATP (or ATP analogues) may be present in a second population of cells, and the reduction step (a) may be preceded by a step of selective liberation of ATP from the second population of cells. In cases where the second population of cells comprises animal cells and the first population comprises bacterial cells, the animal cells can normally be selectively lysed in much milder conditions than the most types of relevant bacterial cells. For instance, a mild detergent such as Triton X-100 may be used to selectively lyse animal cells in the presence of bacterial cells. See for example U.S. Pat. No. 4,303,752.

The second population of cells may be host cells from the animal from which the biological sample is derived.

The method according to the first aspect may also be applied to non-biological samples, such as cell culture medium, food, beverages, pharmaceuticals, sewage, environmental samples such as swabs from environmental surfaces, drinking water and the like.

The method for determining the amount of ATP present in first population of cells in a sample may additionally comprise step (d) of calculating the number or concentration of viable cells of the first population present in the sample from the amount of ATP determined in step (c). Since the ATP concentration in a cell is fairly constant, the number of cells may be estimated from the amount of ATP determined, e.g. by comparing the reading to a standard curve obtained from a similar type of cell.

Methods for Doing Sequencing-By-Synthesis, Such as Pyrosequencing

In general terms, any sequencing-by-synthesis procedure involves sequential addition of a known deoxynucleotide (or an analogue thereof) to a reaction mixture where DNA synthesis will occur dependent on the sequence of the DNA to be sequenced. The addition is followed by determining whether DNA synthesis actually occurred (allowing determination of the sequence), followed by elimination of excess added nucleotide before the next cycle is initiated. It is of importance that the elimination is complete and no intermediate products such as deoxynucleotide diphosphates accumulate, as this would interfere with the subsequent sequencing cycles. The elimination of nucleotides may according to the present disclosure advantageously be performed with SFA, since it exhibits better properties in nucleotide elimination (deoxynucleoside di- and triphosphates) than the commonly used STA (see Example 11). Thus, the method of the first aspect may be a sequencing-by-synthesis procedure, the contaminating nucleotides consist of or comprise excess dNTPs or analogues thereof present after a completed sequencing cycle, and the analysis performed on the sample is a sequence readout.

Pyrosequencing is a well-established DNA sequencing method of the sequencing by synthesis-type, based on the detection of pyrophosphate (PPi) released during DNA synthesis. Briefly, the steps of a typical pyrosequencing protocol can be outlined as follows:
1. A sequencing primer is hybridized to a single stranded DNA template to be sequenced, and incubated with the following enzymes: a DNA polymerase, an ATP sulfurylase, a luciferase and an apyrase, and the following substrates: adenosine 5′ phosphosulfate (APS) and luciferin.
2. The first of four deoxynucleotide triphosphates (dNTP) is added to the reaction. It should be noted that deoxyadenosine alpha-thio triphosphate (dATPaS) is used as a substitute for the natural deoxyadenosine triphosphate (dATP) since it is efficiently used by the DNA polymerase, but not recognized by the luciferase. The DNA polymerase then catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide.
3. ATP sulfurylase quantitatively converts the PPi to ATP in the presence of adenosine 5′ phosphosulfate. The produced ATP drives the luciferase-mediated conversion of luciferin to oxyluciferin that generates visible light in amounts that are proportional to the amount of ATP. The light produced in the luciferase-catalyzed reaction is detected by a detector (such as a CCD-camera). Each light signal is proportional to the number of nucleotides incorporated.
4. Apyrase continuously degrades any unincorporated dNTPs and excess ATP. When degradation is complete, another dNTP can be added to continue with the next cycle in sequencing. Addition of dNTPs is performed one at a time. As the process continues, the complementary DNA strand is built up and the nucleotide sequence can be determined from a graph showing light output over time in relation to the time points of addition of the dNTPs.

Since the commercially available enzymes show batch-to-batch variations in their quality and activity, the applicability of pyrosequencing reaction is limited for sequencing longer oligonucleotides. However, the present invention provides that Shigella flexneri apyrase can be used as the apyrase in a pyrosequencing reaction with improved results, since it is more efficient in eliminating deoxynucleoside di- and triphosphates (see Example 11).

Thus, the present invention provides a method of the first aspect, being a method for performing pyrosequencing, and comprising the steps of:
a. performing a pyrosequencing reaction comprising addition of a nucleoside triphosphate;
b. converting the pyrophosphate released in step (a) into ATP via an enzymatic reaction;
c. determining of the amount of ATP formed in step (b);
d. degrading unincorporated nucleoside triphosphate from step (a) and ATP formed in step (b) with an apyrase enzyme;
e. repeating the steps a-d at least once;
wherein the apyrase enzyme is a Shigella flexneri apyrase.

General Aspects of the Methods

In all of the methods above, the determination of ATP may be performed with a bioluminescent assay. Preferably, the determination of ATP is performed with a bioluminescent assay utilizing luciferin and luciferase. Luciferase may be provided in a composition according to the eigth aspect, to enable inhibition of apyrase concomitant to luciferase addition.

The Shigella flexneri apyrase in the methods mentioned above may comprise an amino-acid sequence having sequence identity of 80%, more preferably 85%, even more preferably 90%, yet more preferably 95%, still more preferably 97% to the sequence according to SEQ ID NO: 10. Importantly, only such sequence deviations from the native Shigella flexneri apyrase according to SEQ ID NO: 10 that retain the apyrase activity are encompassed in the invention. Most preferably, the Shigella flexneri apyrase in the methods above comprises or consists of an amino-acid sequence according to SEQ ID NO: 4. Shigella flexneri apyrase may be provided in a composition according to the seventh aspect of the invention.

Apyrase Reagents and Their Production

In a second aspect, the present invention relates to a Shigella flexneri apyrase comprising an amino-acid sequence with at least 95%, more preferably 96%, yet more preferably 97%, still more preferably 98% identity, even more preferably 99% sequence identity to the sequence according to SEQ ID NO: 4. Importantly, only such sequence deviations from the Shigella flexneri apyrase according to SEQ ID NO: 4 that retain the apyrase activity are encompassed in the invention. Most preferably, the Shigella flexneri apyrase of the second aspect comprises or consists of an amino-acid sequence according to SEQ ID NO: 4.

In a third aspect, the present invention relates to a polynucleotide encoding an apyrase according to second aspect.

In a fourth aspect, the present invention relates to a vector comprising a polynucleotide of the third aspect operably linked to a promoter capable of inducing expression of the polynucleotide, preferably via arabinose induction.

In a fifth aspect, the present invention relates to a host cell comprising a polynucleotide of the third aspect operably linked to a promoter capable of inducing expression of the polynucleotide, or a vector of the fourth aspect.

In a sixth aspect, the present invention relates to a method of producing a recombinant apyrase according the second aspect, comprising the steps of:
culturing host cells according to the fifth aspect in conditions inducing the expression of the polynucleotide; and
recovering the produced recombinant apyrase from the culture.

In a seventh aspect, the present invention provides a composition comprising a Shigella flexneri-apyrase having a pH in the range of 6-9, preferably 7-8, and an ionic strength of at least 300 mM. Preferably, the pH is about 7.5. The buffer may be a HEPES buffer. The ionic strength may be 300-1000 mM, preferably 400-800 mM, more preferably 500-800 mM. The buffer may comprise 400-600 mM NaCI and 20-30 mM MgSO₄. As shown in Example 10, the buffer of the seventh aspect is particularly advantageous in terms of enzyme stability.

In an eighth aspect, the present invention provides a composition comprising a luciferase and an apyrase inhibitor comprising ortho-vanadate. The advantages of ortho-vanadate are discussed in the context of the first aspect of the present invention. It is advantageous to combine the apyrase inhibitor with the luciferase in a single composition, enabling both to be added in a single action. The concentration of ortho-vanadate should chosen such that the final concentration of ortho-vanadate in the sample after addition of the composition to a reaction mixture being used in a luciferase-based assay is sufficient to inhibit SFA. The concentration of ortho-vanadate may be at least 0.2 mM, preferably at least 1 mM, more preferably 1-3000 mM, most preferably 2-200 mM. The amount of luciferase activity should be sufficient for a luciferase-based assay after addition of the composition to a reaction mixture.

Uses of SFA Apyrase

In a ninth aspect, the present invention relates to a use of a Shigella flexneri apyrase for degrading contaminating nucleotides (nucleotide triphosphates and/or nucleotide diphosphates) in an analytical method. The contaminating nucleotides may be ATP and/or ATP analogues, and the analytical method may be an assay for measuring ATP. The assay for measuring ATP may be a bioluminescent assay.

In an tenth aspect, the present invention relates to a use of a Shigella flexneri apyrase in a DNA sequencing reaction, preferably a sequencing-by-synthesis reactions, most preferably a pyrosequencing method.

The apyrase in the ninth or the tenth aspects may comprise an amino-acid sequence having sequence identity of 80%, more preferably 85%, even more preferably 90%, yet more preferably 95%, still more preferably 97% to the sequence according to SEQ ID NO: 10. Importantly, only such sequence deviations from the native Shigella flexneri apyrase according to SEQ ID NO: 10 that retain the apyrase activity are encompassed in the invention. More preferably, the apyrase in the ninth and the tenth aspects may be an apyrase according to the second aspect.

All references cited herein are hereby incorporated by reference. The arrangement of the present disclosure into sections with headings and subheadings is merely to improve legibility and is not to be interpreted limiting in any way, in particular, the division does not in any way preclude or limit combining features under different headings and subheadings with each other.

The following examples are not to be construed as limiting of the invention.

### EXAMPLES

For experimental details concerning the examples below, the reader is directed to a separate section titled Materials and Methods.

### Example 1

### Production and Colorimetric Evaluation of Recombinant Shigella flexneri Apyrase (SFA)

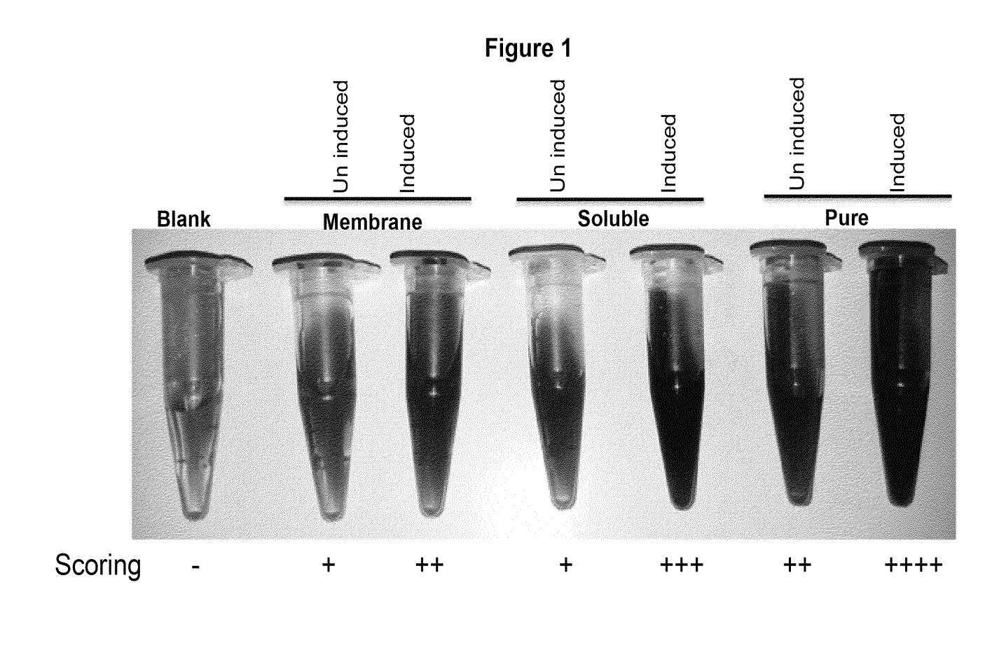

The SFA was produced and purified as described in the materials and methods section. The purified enzyme fraction was examined using a colorimetric assay to identify the presence of bacterial apyrase. As can be seen in FIG. 1, the colorimetric assay clearly shows the color difference that directly corresponds to expression levels of apyrase enzyme expression, thus revealing its cellular location. In all of the cases, a clear difference in the color can be seen between non-induced and induced cultures in comparison with the color of the blank sample.

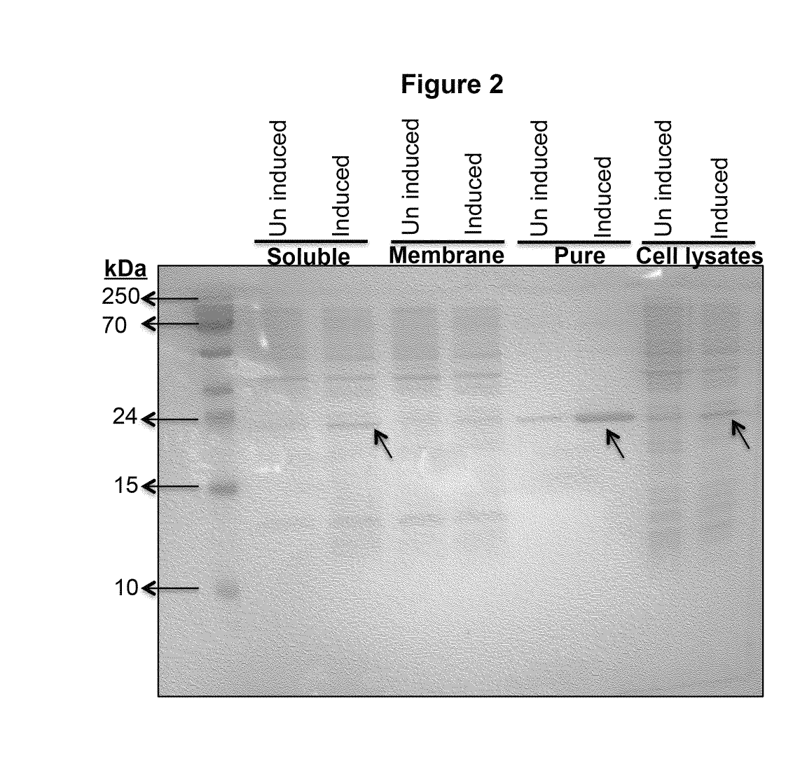

As shown in FIG. 2, a clear correlation can be drawn between the colorimetric results and apyrase expression profiles of the clones and their localization. The enzyme, rSFA was constantly overexpressed in induced cultures at 25 kDa, especially in whole cell lysates and soluble fractions, but not in membrane fractions, thereby indicating its location for the further purification procedures. The induced cultures revealed an expression of ^{˜}10 times more enzyme compared to that of un-induced cells, which demonstrated the efficiency of the constructed clone. This SDS-PAgel also shows the purity of the apyrase production and this purified fraction was used for all the experiments of this study.

### Example 2

### Sequence Analysis Between rSFA and STA

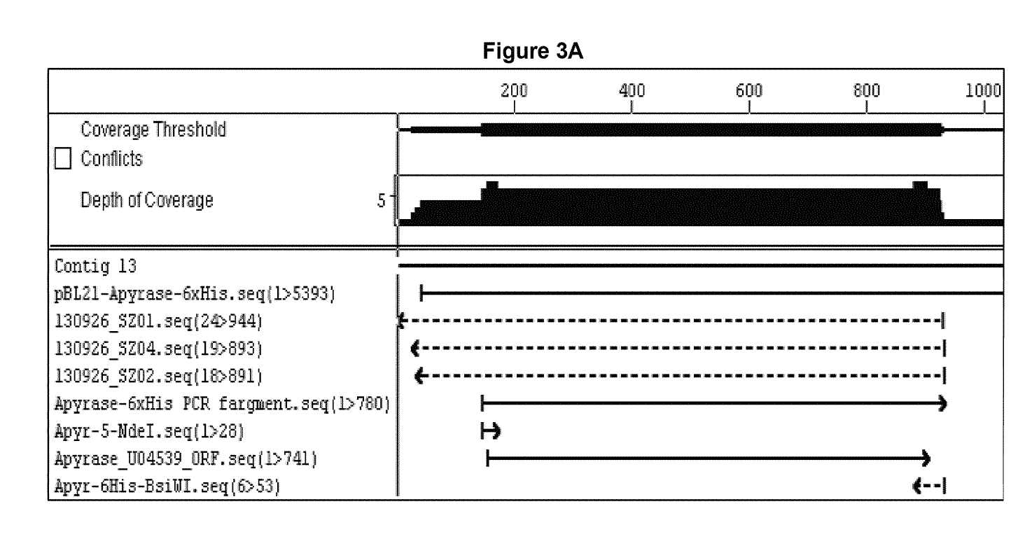

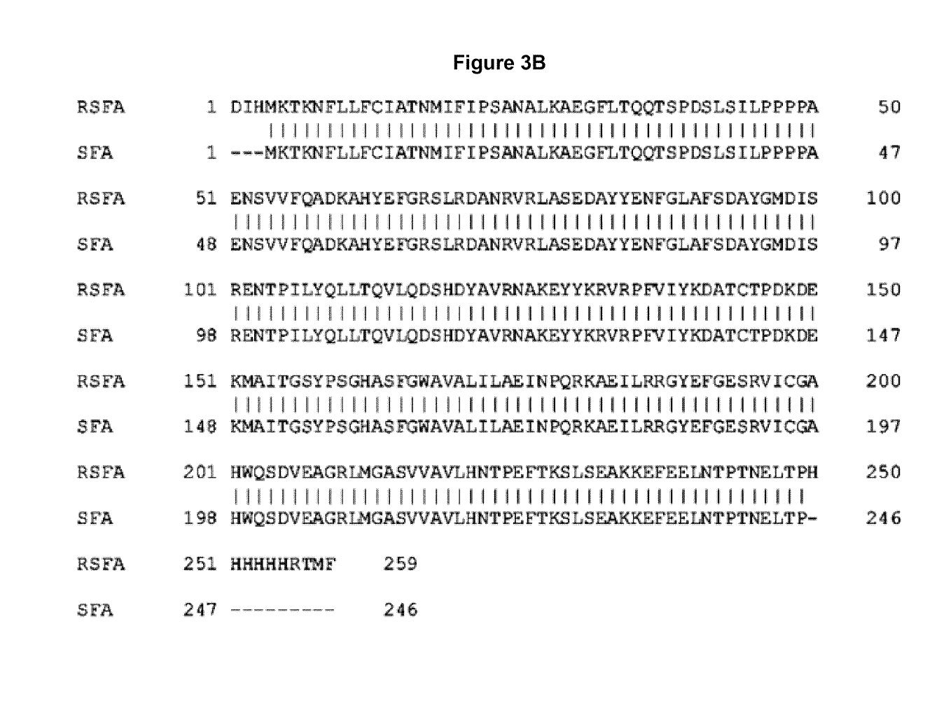

Once the protein expression was confirmed by gel electrophoresis and colorimetric assay, the rSFA gene product was sequenced to identify the nucleotide sequence in order to compare with the reference sequence WP_010921592.1 obtained from the NCBI database (FIG. 3A). As can be seen in the figure, the cloned oligonucleotide fragment of SFA containing Apyrase-6×His (rSFA) and the expressed sequence (pBL21-Apyrase-6×His) sequences fell in the desired range (see FIG. 3A). As can be seen in FIG. 3B, the rSFA differs from the reference SFA only at N- and C-termini.

### Example 3

### rSFA is More Efficient in ATP-Degradation Compared to STA and is Stable

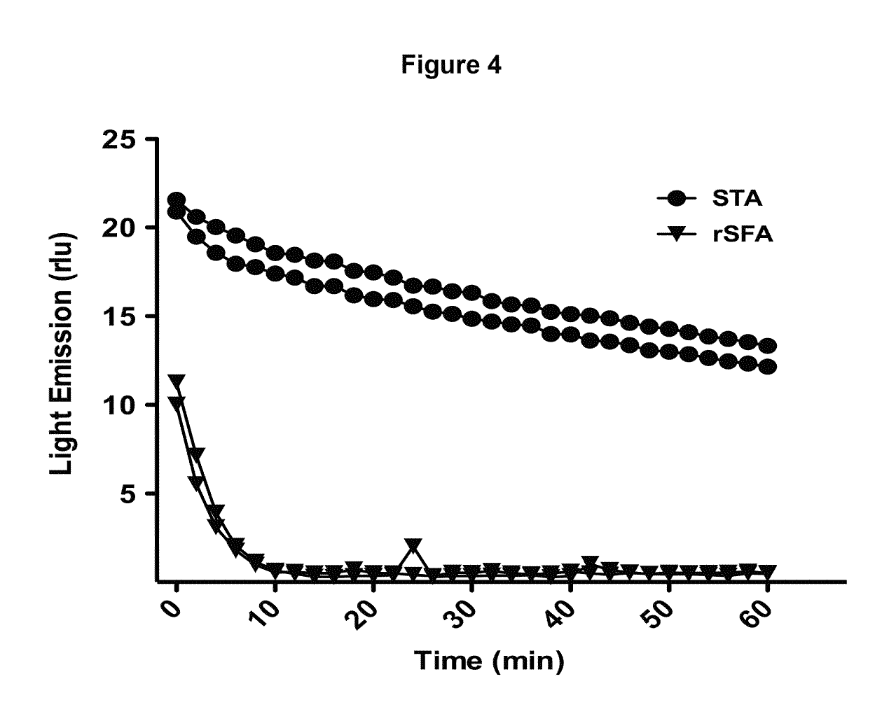

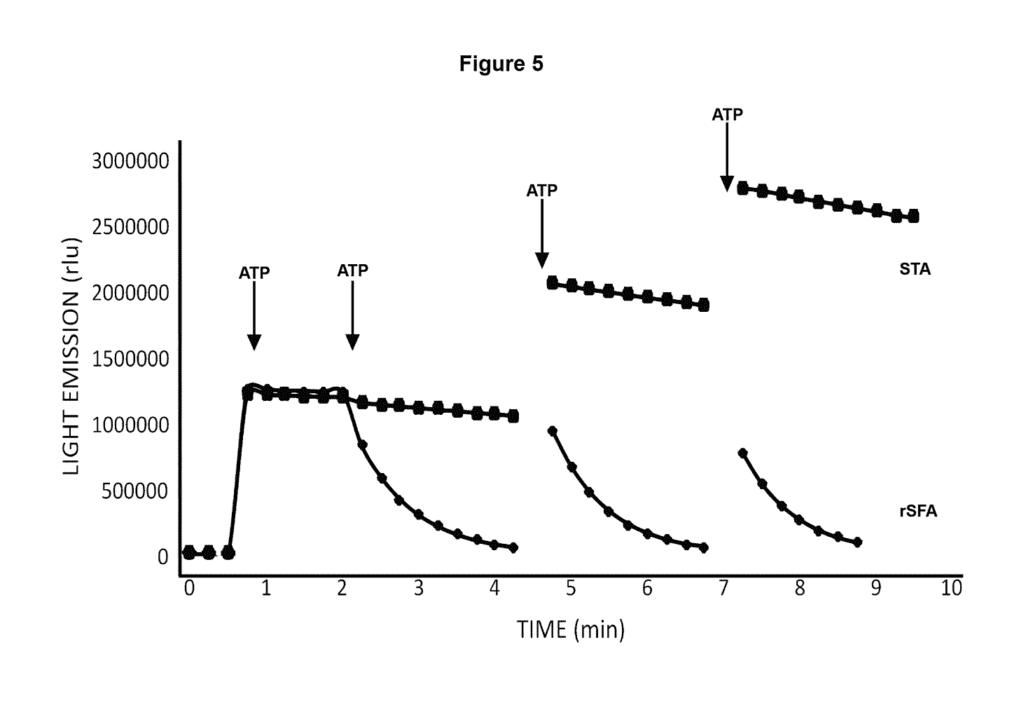

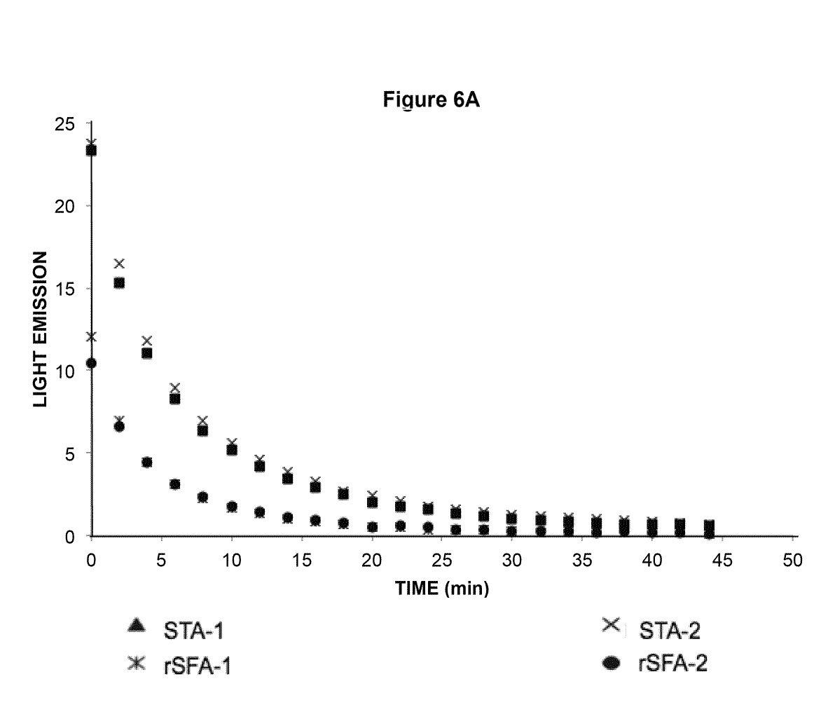

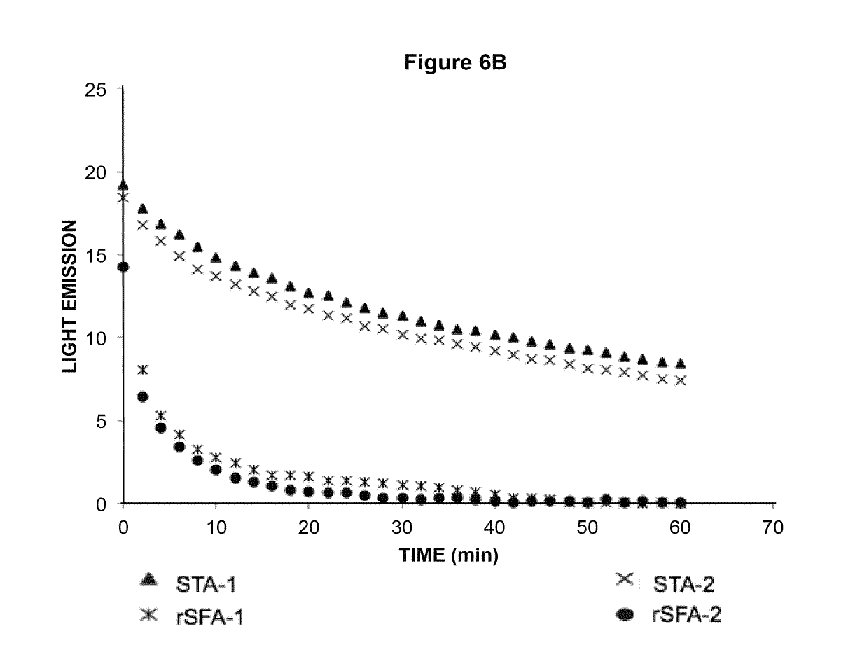

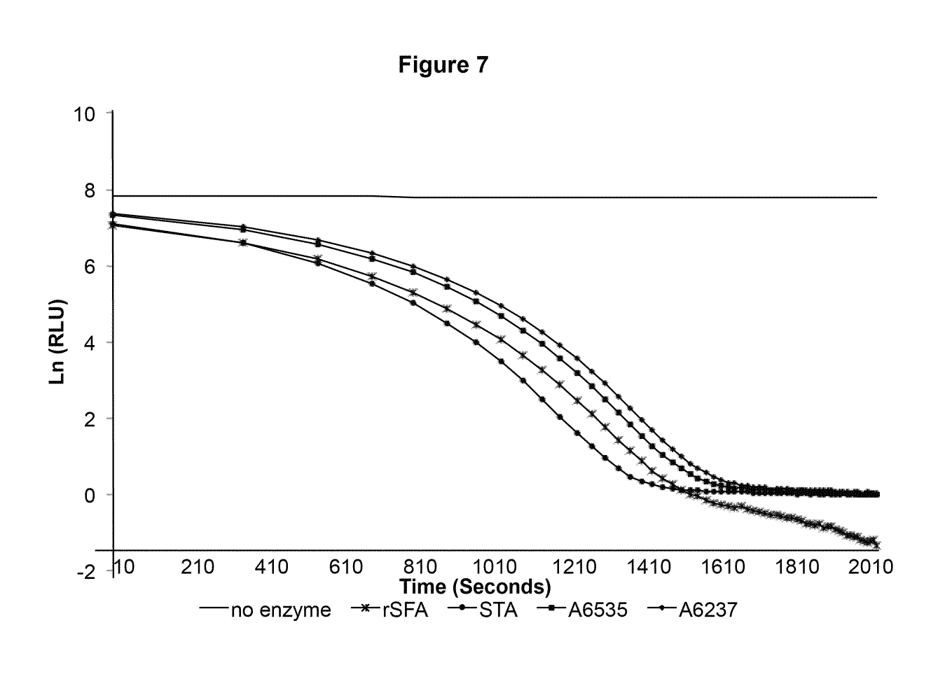

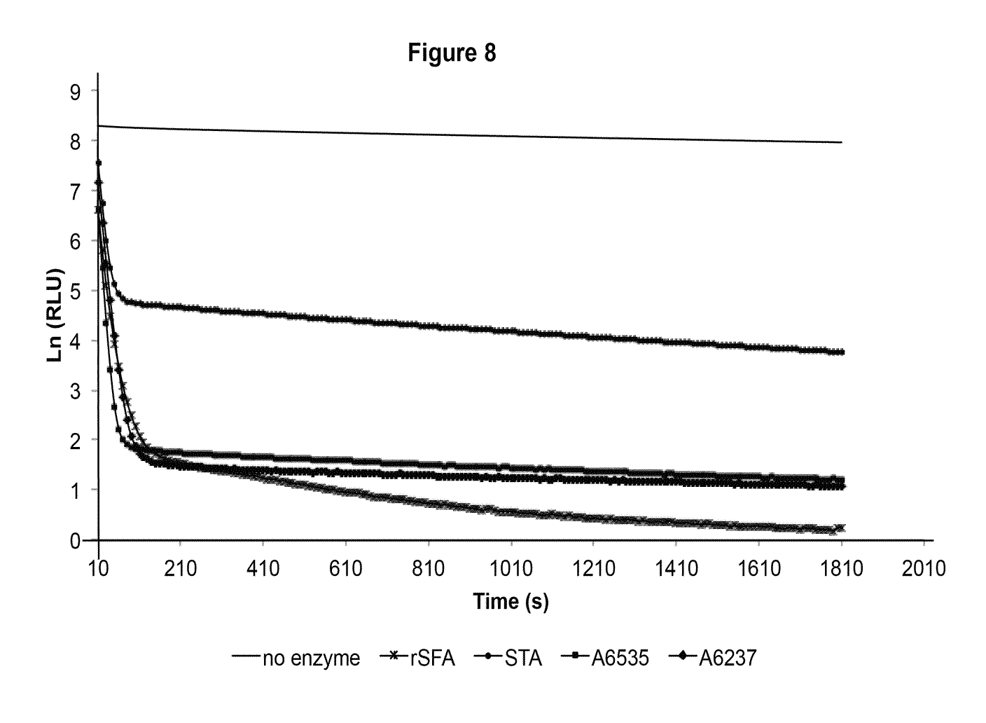

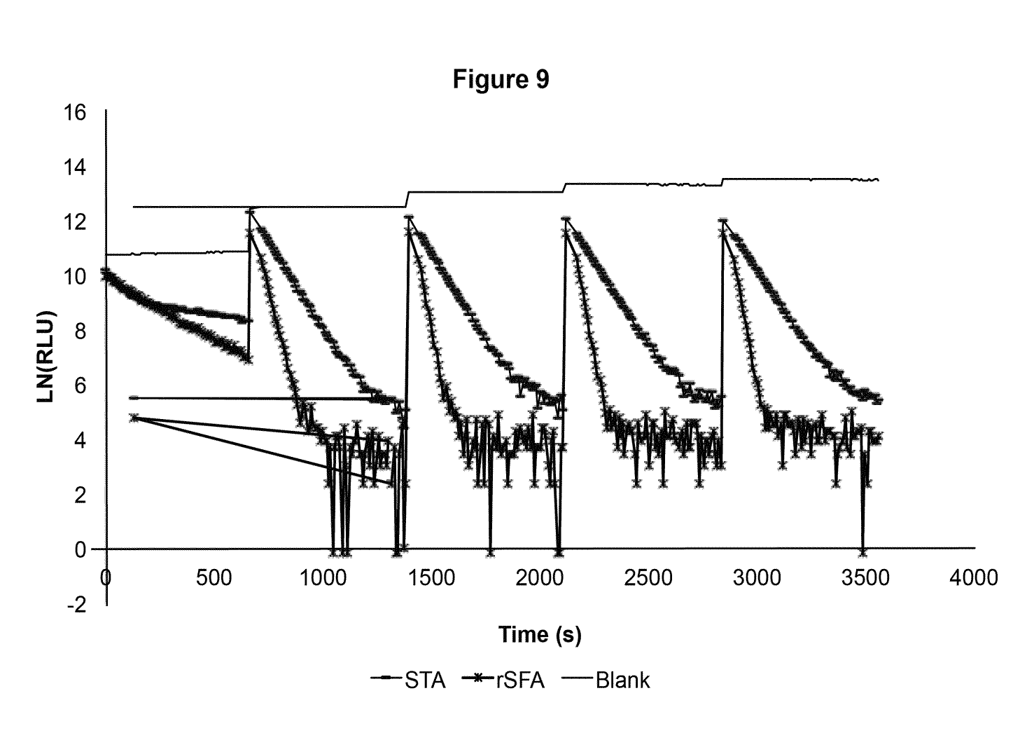

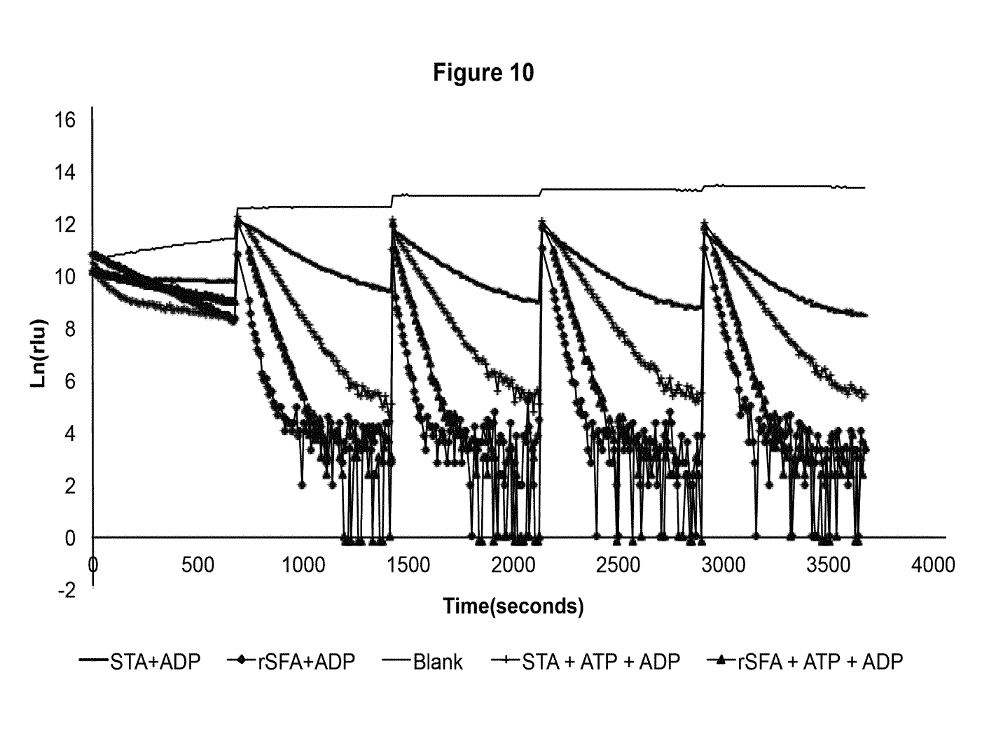

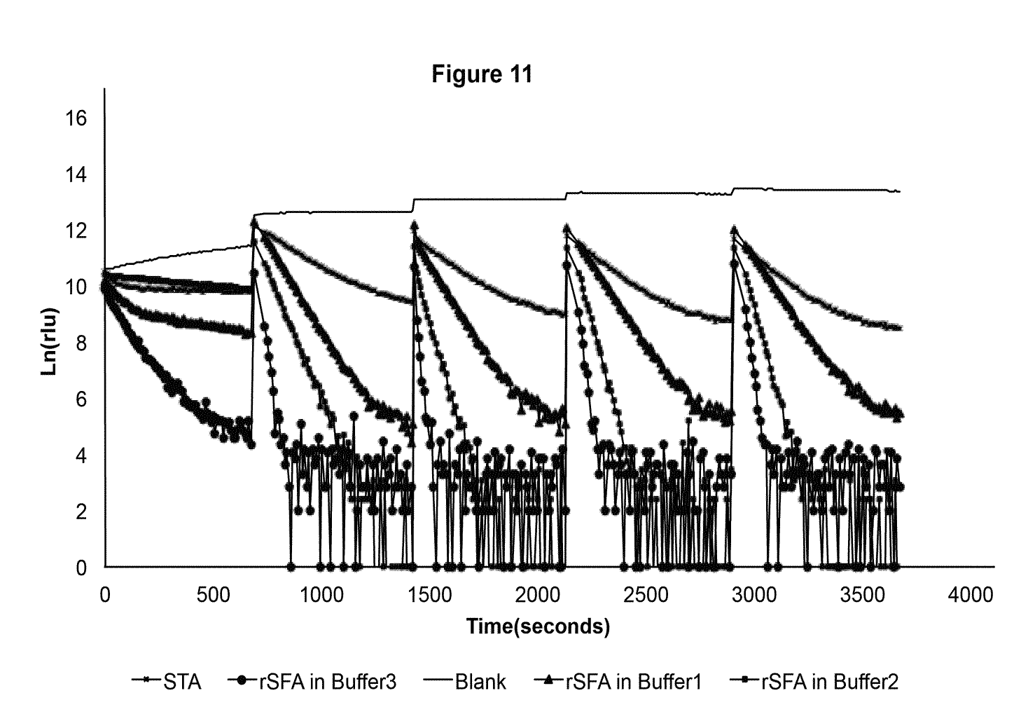

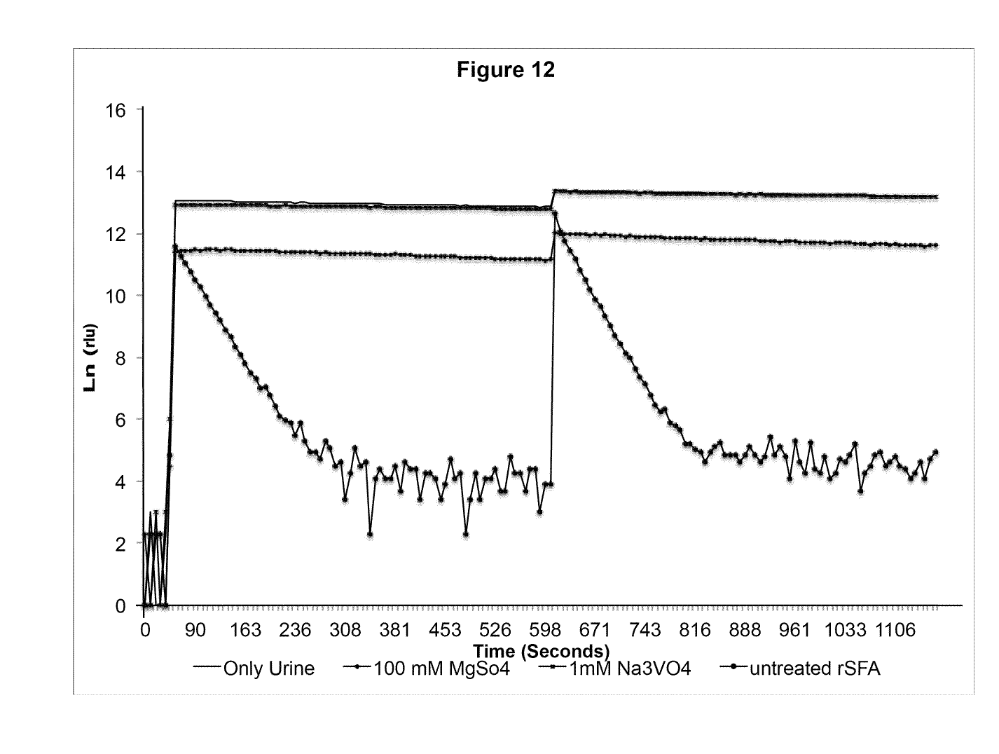

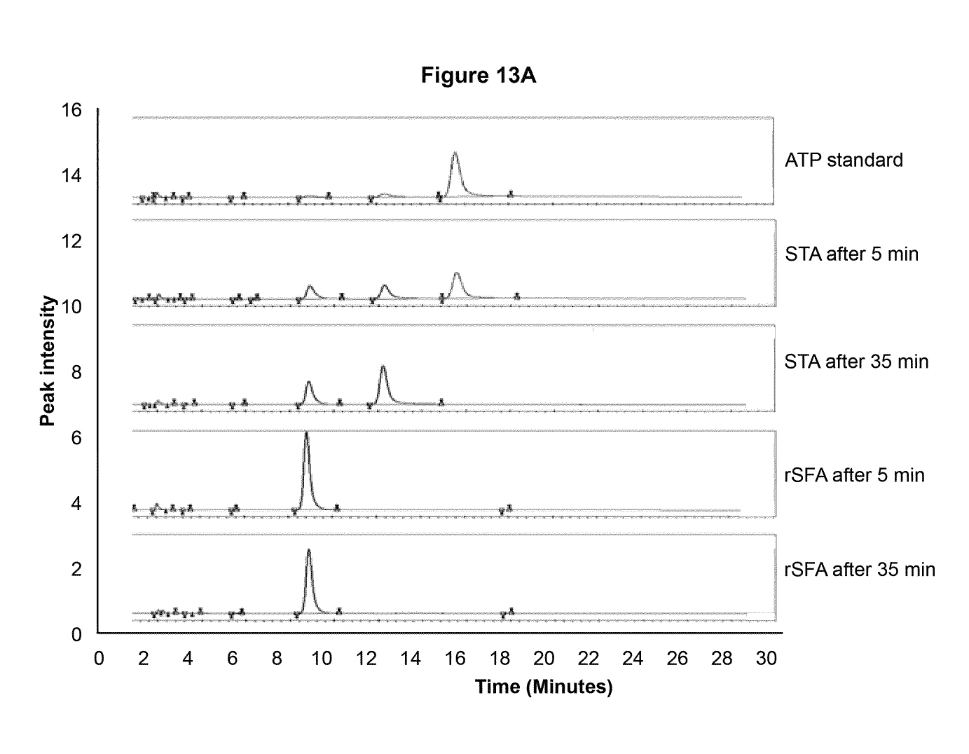

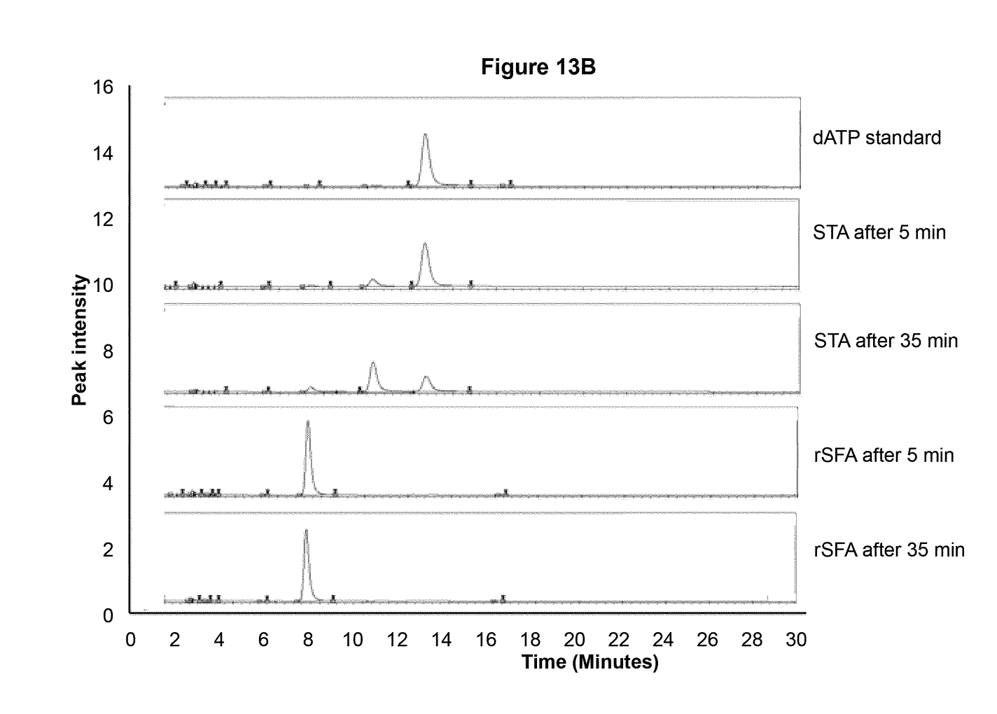

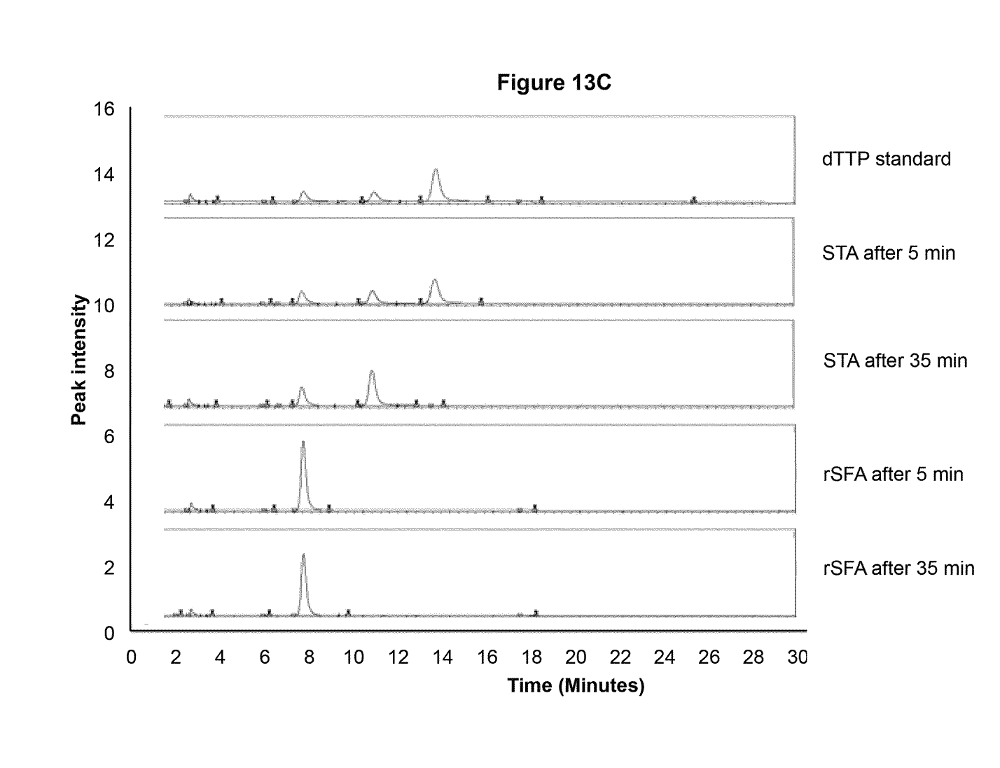

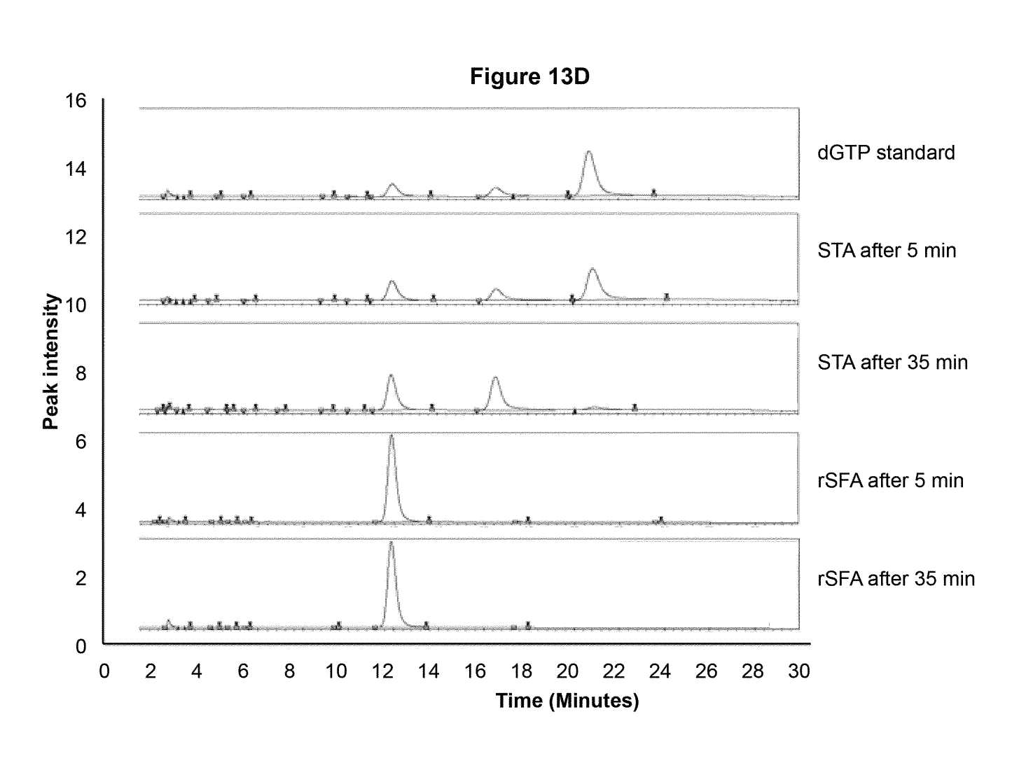

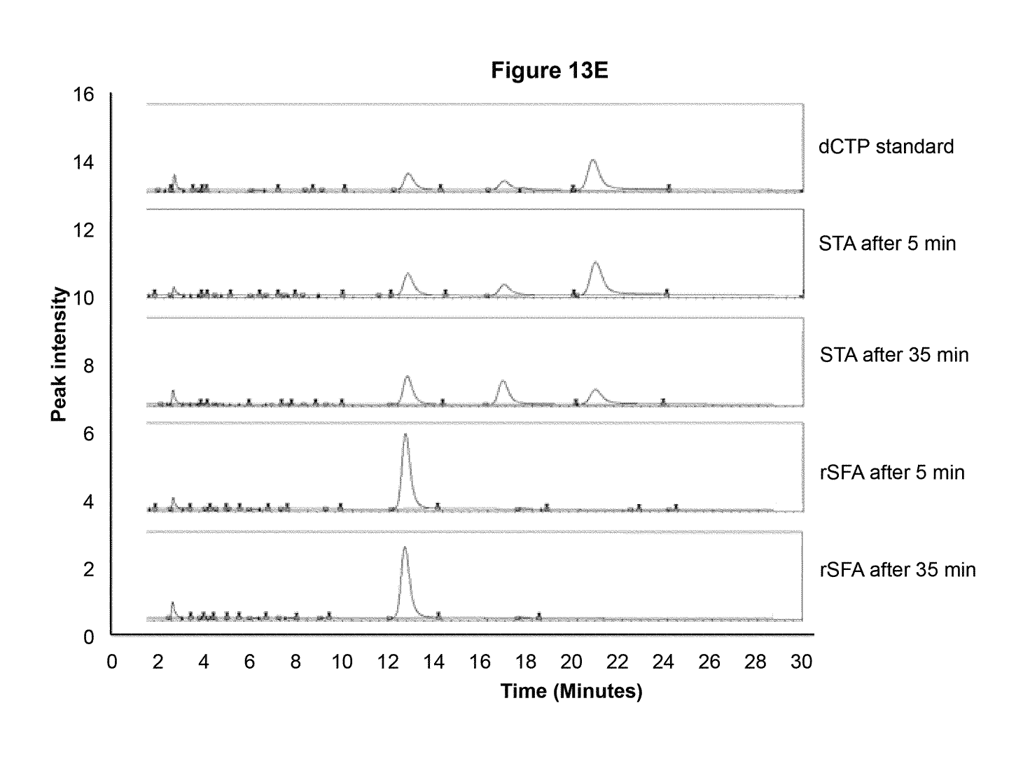

The measurements of the decay of the light emission from the firefly luciferase reaction (FIG. 4) described the ATP-degradation activity between the two apyrases, rSFA and STA. The rSFA reveals a clear elimination of ATP and its analogues in ^{˜}10 min compared to that of STA. In the other experiment, as can be seen in FIG. 5, luminescence was increased upon the addition of ATP in both cases, while, addition of apyrase resulted in a decay of the light because of ATP depletion. Deliberate addition of extra ATP during the reaction revealed a similar ATP depletion trend. However, the rSFA, exhibited more rapid ATP-depletion activity than did the commercially available STA. When ATP as well as the apyrase preparations was diluted 10-fold, the ATP depletion was reduced accordingly.

The rSFA samples were kept at 4° C., then frozen and freeze-thawed to check its stability. An effort was made to see if the rSFA could be used to prepare a reagent for the depletion of extraneous ATP in biological samples.

TABLE 1
ATP-degradation activity comparison between recombinant
Shigella flexneri apyrase and commercially available
potato apyrase procured from Sigma-Aldrich

| | Initial | | | |
|---|---|---|---|---|
| | activity | Remaining ATP activity | | |
| | (Units/mg | | after | after |
| Type of apyrase | protein) | after 1 min | 17 min | 91 min |
| Potato apyrase (STA) | 4.12 | 1.1876% | 0.0367% | 0.0228% |
| Recombinant Shigella | 3.39 | 2.6953% | 0.0019% | 0.0005% |
| apyrase (rSFA)* | | | | |
| Recombinant Shigella | 3.58 | 2.1855% | 0.0019% | 0.0004% |
| apyrase (rSFA)** | | | | |

*Stored frozen before experiment
**Stored in refrigerator before experiment

Furthermore the remaining ATP was much lower after longer incubation times as shown in the Table 1. This indicates that the SFA, is more sensitive and efficient than STA in ATP depletion, as ADP accumulates in the reaction mixture and so hinders the reaction efficiency of the latter.

### Example 4

### Demonstration of Extracellular ATP and its Analogues in Serum

Considering the advantages of rSFA from the above experiments, we checked its efficiency removing extracellular ATP in serum samples. In the first experiment (FIG. 6A), rSFA revealed complete hydrolysis of ATP and its analogues ^{˜}17 min, whereas STA took ^{˜}45min, against 100% crude serum. The linearity was reproduced (FIG. 6B), even when the serum was diluted to 10%. Hence rSFA is superior to that of STA in eliminating ATP in biological samples like serum.

TABLE 2
The effect of serum on ATP depletion rate in crude and diluted serum

| | No serum | Undiluted serum | 10% |
|---|---|---|---|
| | (Negative control) | (100% crude serum) | Diluted serum |
| potato apyrase | 3.52 | 0.55355219 | 2.42904382 |
| recombinant | 3.98 | 1.81641444 | 1.77443406 |
| apyrase | | | |

### Example 5

### Improved Method for Pyrosequencing

Since the commercially available enzymes show batch-to-batch variations in its quality and activity and inability to hydrolyze excess of ATP and its analogues, it decreases the efficiency and limits the applicability of pyrosequencing reaction to read long sequences. This can be addressed using rSFA because it shows better quality and efficiency compared to STA. See also Example 11.

### Example 6

### rSFA is Superior to STA in an ATP Bioluminescent Assay, In Buffer

The time course of activities, including initial and end-point activities of three commercial products of potato apyrase termed STA, A6535 and A6237 were compared with rSFA in a bioluminescent ATP assay. The enzymes were diluted in a buffer, so no sample interference should be present and the results represent an ideal scenario. The measurement was light output from a luciferase, and the desired result is as fast and complete as possible elimination of ATP by the apyrase.

As shown in FIG. 7, the STA variant A6237 showed better activity compared to the other commercial STA sources. Nevertheless, all the commercial STA variants were saturated after 120 s and remained ineffective in further reducing the ATP concentration thereafter. In contrast, rSFA was active in eliminating the ATP-analogues and its by-products even after 300 s and is capable of a more complete elimination of ATP. Note the log scale in the graph, indicating at least 10-fold improvement.

### Example 7

### rSFA is Superior to STA in an ATP Bioluminescent Assay, in 10% Serum

An experiment similar to Example 8, but with 10% serum present in the assay mixture was undertaken to test utility of rSFA in samples containing serum, such as blood samples.

As depicted in FIG. 8, activity of the commercial STA variants (STA, A6535 and A6237) did not show further ATP elimination after 6 min but rSFA continued to cleave ATP-analogues and its by-products even after 30 min. Presence of extracellular ATP could potentially inhibit the activity of STA, whereas rSFA remains active in removing extracellular ATP from 10% serum. Thus, rSFA is superior to STA in the analysis of blood samples.

### Example 8

### rSFA is Superior to STA in an ATP Bioluminescent Assay, In Urine

Considering the complications due to contaminants in urine, the inventors tested the ATP-elimination activities of STA and rSFA for 60 min in an urine sample. Though both the enzymes showed similar initial activities, rate of action of STA was hindered after 3 min, whereas rSFA continued with ATP-elimination. Even after 4 subsequent injections of 10 μL 10 μmol/L ATP, the rSFA showed a complete ATP elimination activity each time in less than 3 min but STA could not digest the ATP even after 20 min (FIG. 9).

### Example 9

### Effect of ATP-Analogues on the Activities STA and rSFA

Hydrolysis of ATP, ADP and their mixtures in urine was tested with STA and rSFA. FIG. 10 shows that the activity of STA was considerably decreased either against only ADP 10 μmol/L or when a mixture of ADP and ATP was supplemented at 10 μmol/L concentrations. In contrast, there was no substantial difference in the activity with rSFA. In both the cases, the STA's phosphate removal activity from ATP-analogues was significantly lower compared to that of rSFA.

### Example 10

### Optimization of Buffer System to Improve the Activity of rSFA

Selection and optimization of a correct buffer system is an important aspect of enzyme stability. The inventors have developed three buffer systems to optimize the stability of rSFA. Equal concentration of rSFA was stored in Buffer 1, Buffer 2 and Buffer 3 for about 10 months at +4° C. and the bioluminescent ATP-elimination assays were performed. The activity of the enzyme was assayed and it was found that the enzyme used with Buffer 3 showed particularly good activity compared with other two buffers (FIG. 11).

### Example 11

### Activity of rSFA on ATP, dATP, dTTP, dGTP and dCTP

To compare rSFA activity on nucleotides to that of STA, ATP, dATP, dTTP, dGTP and dCTP were incubated with the enzymes (same initial activity) for 5 min and 35 min and the resulting products were detected using high performance liquid chromatography (HPLC).

As a starting point, chromatographic analysis of ATP (FIG. 13A) revealed that it is only partially cleaved to ADP and AMP in 5 min by STA, and all of ATP was completely converted into ADP and AMP after 35 min. In contrast, rSFA fully converted all ATP into AMP in less than 5 min with no detectable ADP remaining.

FIG. 13B shows that more than 25% of dATP was remaining after 35 min of incubation with STA (product mainly dADP, and some dAMP), whereas rSFA fully converted the dATP into dAMP in less than 5 min.

Similarly, dTTP was converted in to TDP and TMP in 35 min by STA, whereas rSFA fully converted dTTP it into TMP in less than 5 min (FIG. 13C).

Similar results were observed with dGTP (FIG. 13D).

However, as can been in FIG. 13E, the STA could convert only half of the dCTP into CDP and CMP after 35 min,whereas rSFA completely converted the dCTP into CMP in less than 5 min. Therefore, rSFA is at least 7 times more potent in eliminating dCTP than STA.

The above results indicate that SFA is superior to STA for eliminating dNTPs and dNDPs, which is in particular relevant for use in a sequencing-by-synthesis method (such as pyrosequencing), since the accumulation of dNTP and/or dNDPs interfere with subsequent sequencing cycles.

### Example 12

### rSFA Can Be Inhibited by Ortho-Vanadate Without Interferering with Luciferase Activity

The activity of rSFA may in some cases need to be stopped in order to measure the intracellular concentration of ATP. Among the different inhibitors tested, 100 mM of MgSO₄ and 1 mM of ortho-vanadate in the form of Na₃VO₄, where only the former affected the activity of luciferase (FIG. 12). The ortho-vanadate did not affect luciferase and at the same time, it completely inhibited the activity of rSFA, making it ideal for inhibiting apyrase in an assay involving luciferase.

Materials and Methods

Cloning, Sequencing and Production of Shigella apyrase

The pRSETB plasmid bearing Shigella flexneri 2a apy gene in E. coli GJ1158 strain (Bhandari and Gowrishankar J Bacteriol. 1997 Jul;179(13):4403-6) was obtained from Sankaran, Centre for Biotechnology, Anna University, India. Since the GJ1158 construct was not stable, the native apy gene was excised and amplified (using apy 3 and apy 4 primers; Table 3), which was further purified by PCR gel purification Kit (Fermentas). The purified DNA template was introduced with two-endonuclease restrictase sites for NdeI and BsiWI to replace natural stop codon by 6-Histidine-coding region using the primers Apyr-6His-BsiWI and Apyr-5-NdeI (Table 2) and transformed in E. coli TOP10 (Invitrogen) according to the manufacturer's instructions with minor modifications. Transformants were grown on agar plates and the positive pBL-Apyrase-6His clones were selected by NheI hydrolysis and size verification of restriction fragments on agarose gels, followed by sequence verification at Karolinska Institute's sequencing facility, Stockholm. Identified sequences of pBL-Apyrase-6His constructs were compared with Shigella flexneri 2a apyrase (GeneBank accession: U04539) to find out the integrity of cloning sites, Apyrase-6His coding sequence and replacement of the stop codon by 6-His tag. After sequence confirmation, the plasmid of pBL-Apyrase-6His clone (SEQ ID NO: 3) was transformed in E. coli BL21-A1 for the protein expression. The amino-acid sequence encoded by the plasmid is shown in SEQ ID NO: 4.

TABLE 3
List of oligonucleotides used

| Name | Oligonucleotide sequence (5′→3′) | SEQ ID NO |
|---|---|---|
| Native apyrase | CGCGGATCCCTGAAGGCAGAAGGTTTTC | SEQ. ID NO: 5 |
| primer1 | | |
| Native apyrase | CCCAAGCTTTTATGGGGTCAGTTCATT | SEQ. ID NO: 6 |
| primer2 | | |
| Apyr-6His- | GAACATCGTACGTTAGTGGTGATGGTGATGATG | SEQ. ID NO: 7 |
| BsiWI | TGGGGTCAGTTCATTGGTAG | |
| Apyr-5-Ndel | GATATACATATGAAAACCAAAAACTTTC | SEQ. ID NO: 8 |

Large-Scale Production of Pure Shigella Apyrase

Preliminary evaluation using Ni-NTA magnetic beads: About 1.3 ml of bacterial culture was pelleted at 13,000 rpm and 300 μl of extraction buffer (1 mM PMSF, 1 mg/ml lysozyme and 0.05% Triton X-100 in 20 mM Tris-HCl, pH 8.0) to prepare cell lysates. After 30 min, 30 μl of equilibrated Ni-NTA magnetic beads were added and kept on mild agitation to facilitate the binding of His-tagged protein to the beads. Supernatant containing the soluble proteins was sampled and the beads were washed with 300 μl of washing buffer (300 mM NaCl, 20 mM imidazole, 8% glycerol and 0.2% Triton X-100 in 10 mM KP, pH 7.8). After rinsing the beads with PBS for 10 min, they were boiled for 3 min with 12 ul of SDS to elute pure recombinant protein. The protein profiles of cell lysates, soluble proteins and pure protein fractions were resolved on 12% SDS-PA gels at 30 mA using Tris-glycine (25 mM Tris-HCl, 0.1% w/v SDS, pH 8.3). After confirmation, apyrase production was scaled-up to large scales up to 2 L.

Large-scale purification by metal affinity chromatography: The apy gene (SFA) bearing E. coli BL21-A1 strain containing pBL-Apyrase-6His was cultured in 2 L of LB medium and induced at OD 0.7. Cells were harvested by centrifuging at 4000 rpm after 4 h of growth and after a saline wash they were lysed by French press. The 6-histidine-tagged SFA was purified by metal affinity (IMAC) (HisTrap FF, GE-Healthcare, Sweden) using a buffer (20 mM Hepes, 500 mM NaCl, 10% glycerol, 20 mM imidazole, pH 7.5) and after washing with a wash buffer containing 50 mM imidazole, the enzyme was eluted with elution buffer containing 350 mM imidazole. The buffer was exchanged with buffer containing 25 mM MgSO₄, 5% BSA and the protein sample was concentrated using an ultrafiltration device with a 10 kDa cut-off semi-permeable membrane (Vivaspin Turbo, Sartorius).

Colorimetric Evaluation of Apyrase Activity

The enzyme activity was evaluated by calorimetrically, as described by Sankaran et al. (Diagn Microbiol Infect Dis. 2009 March;63(3):243-50) with minor modifications. The colorimetric reaction was performed by mixing equal volumes of assay reagent (6M sodium pyrophosphate and 40 mM EDTA; pH 7.5) and a color reagent (5% w/v acidic ammonium molybdate and 1% w/v ferrous ammonium sulfate) in the presence of apyrase enzyme (test sample) to produce colorimetric signals, which are proportional to the concentration of enzyme present in the test sample. Principally, the assay uses inorganic pyrophosphate as a substrate for the enzyme, while EDTA acts both as a buffer and a metal chelator to suppress the other contaminating phosphatase and pyrophosphatase activities. The released phosphate is measured in the presence of pyrophosphate using acidic ammonium molybdate and the color was measured at 695 nm in a multi-mode microplate reader (SpectraMax® M5, Molecular Devices, LLC, USA).

Sensitivity Tests by Luminescent Experiments

Cuvettes contained 100 μL sample in a total volume of 1 mL containing ATP Reagent SL (BioThema AB, Sweden) in 0.07 mol/L Tris- EDTA Buffer and the reaction was started by injection of 10 μL 10 μmol/L ATP Standard. The initial apyrase activity was determined as described by Lundin and coworkers (Analytical Biochemistry 75, 611-620 (1976) and Methods in Enzymology vol 305, 346-370 (2000)) with some modifications. Bioluminescence was measured at every 10 s during 1 min, in the fully automatic 1251 Luminometer (LKB-Wallac, Turku, Finland). To this, 10 μl of 10 μmol/L ATP was added to determine the background ground luminescence. The temperature control was set to 25° C. and bioluminescence was measured from several cuvettes in parallel during 30 or 60 minutes and compared to residual ATP after various incubation times using rSFA and STA. This was repeated with rSFA and STA and a graph was plotted by keeping the ATP-degradation rate as internal control to determine the ATP-degradation efficiency of both of the enzyme sources.

Bioinformatic Analysis of Different Apyrases (ATP-diphosphohydrolase)

The DNA sequence of Shigella flexneri 2a apyrase (GI 532975) (SEQ ID NO: 2) and of the translated potato apyrase (Translated Gene ID 1025774 of Solanum tuberosum ATP-diphosphohydrolase NM_008XM_004845) (SEQ ID NO: 1) sequences were obtained from the NCBI nucleotide database. These nucleotides were analyzed using online tools indicated above for the homology search, multiple gene alignments and their translations to protein sequences.

Optimization of Buffer System

Equal concentration of rSFA was stored in Buffer 1(20 mM Tris, 1 mM EDTA, 1% BSA, 25 mM MgSO₄, pH 7.5), Buffer 2 (20 mM Tris, 25 mM MgSO4, 2.5% BSA, pH 7.5) and Buffer 3 (20 mM HEPES, 500 mM NaCl, 25 mM MgSO₄, pH 7.5) for about 10 months at 4° C. Bioluminescence activity assay was performed as described below to detect the activity of rSFA after storage.

Inhibitor Development

After checking the rSFA inhibitory activity in buffer, it was verified with urine, where 10 μl were added with 80 μl of cell lysate reagent and 10 μmol/L of ATP. About 100 mM of MgSO₄ and 1 mM of Na₃VO₄ were added in separate tubes and incubated for 10 min before measuring the light emission using firefly luciferase.

Bioluminescence Assay

Light emission from the firefly-luciferase reaction was measured at 25° C. in a 1251 Luminometer (LKB-Wallac; Finland) using the ATP Kit SL (BioThema AB, Sweden) according to the manufacture's instruction (in buffer experiment) or adding concentrated luciferase (in serum/urine experiments). Initially luciferase reaction follows first-order kinetics and the ATP Reagent SL in this kit produces light intensity proportional to the ATP concentration. A plot of the natural logarithm of the light intensity versus time yields a straight line with the slope representing the rate constant of the reaction. Starting concentration of STA, A6535, A6237 (Sigma-Aldrich) was adjusted similar to rSFA. BL-reaction was started automatically upon addition of 0.2 μmol/L ATP, and the light intensity was measured every 10 second for 10 minutes (in buffer experiment) or 30 minutes (in serum and urine experiments), in the reaction volume of 0.5 mL.

High performance liquid chromatography (HPLC) analysis of nucleosides/nucleotides

The dNTP Standard preparation was prepared to contain final concentration of 0.1 mM enzyme. Nucleosides ATP/dATP/dCTP/dGTP/dTTP were suspended in Tris-EDTA buffer and diluted 10 times with the mobile phase (70% acetonitrile, 30% ammonium acetate pH 5.35). The above-prepared nucleosides were individually incubated with equal amounts of rSFA and STA for 35 min at pH 7.75 and 5 μl of the mixture was injected into HPLC instrument (SeQuant ZIC®-pHILIC column, Merck Millipore) at a flow rate of 0.7 mL/min using a pump (Jasco PU-2089 Plus). Peaks were detected at 254 nm by UV-2075 Plus detector and the chromatographic separation was achieved using isocratic elution. Analysis was performed using Chrompass software, where each nucleotide/nucleoside was identified by comparison with retention time of the respective standards.

1-39. (canceled)

40. A method, comprising:
providing a sample containing contaminating nucleoside diphosphates nucleoside triphosphates, or both, such as ATP, ATP analogues, or both including deoxyribonucleoside triphosphates;
reducing the amount of the contaminating nucleoside diphosphates, nucleoside triphosphates, or both in the sample with an apyrase enzyme, wherein said apyrase enzyme is a Shigella flexneri apyrase; and
performing an analysis of the sample, wherein said analysis comprises an assay that would have been affected by the contaminating nucleoside diphosphates, nucleoside triphosphates, or both had they not been reduced.

41. The method according to claim 40, further comprising determining the amount of ATP in the sample, comprising:
reducing the amount of contaminating ATP, ATP analogues, or both in the sample by degradation with an apyrase enzyme, wherein the apyrase enzyme is a Shigella flexneri apyrase;
making the ATP to be determined available for determination; and
determining the amount of ATP in the sample.

42. The method according to claim 41, wherein the method further comprises determining the amount of ATP present in a first population of cells in the sample, comprising:
reducing the amount of contaminating ATP, ATP analogues, or both in the sample by degradation with an apyrase enzyme, wherein the apyrase enzyme is a Shigella flexneri apyrase;
liberating the ATP to be determined from the first population of cells; and
determining the amount of liberated ATP from the first population of cells.

43. The method according to claim 42, wherein liberating the ATP to be determined from the first population of cells involves lysis of the first population of cells.

44. The method according to claim 42, wherein the first population of cells comprises bacterial cells.

45. The method according to claim 42, wherein the first population of cells comprises mammalian cells.

46. The method according to claim 42, wherein the sample is a biological sample from an animal.

47. The method according to claim 46, wherein the sample is a biological sample from a human.

48. The method according to claim 40, wherein the sample is a blood sample, a plasma sample, a serum sample, a urine sample, a fecal sample, or a swab from a patient.

49. The method according to claim 41, wherein at least a fraction of the contaminating ATP is present in a second population of cells, and reducing the amount of contaminating ATP, ATP analogues, or both in the sample is preceded by selective liberation of ATP from the second population of cells.

50. The method according to claim 49, wherein the second population of cells are host cells from an animal from which the sample is derived.

51. The method according to claim 40, where the method comprises adding an apyrase inhibitor after reducing the amount of the contaminating nucleoside diphosphates, nucleoside triphosphates, or both in the sample.

52. The method according to claim 51, wherein the apyrase inhibitor comprises ortho-vanadate or Mg2+.

53. The method according to claim 52, wherein ortho-vanadate is present at a concentration of at least 0.1 mM.

54. The method according to claim 40, wherein the sample is a cell culture medium sample, food sample, a beverage sample, a pharmaceutical sample, a sewage sample, an environmental sample such as a swab from a surface, or a drinking water sample.

55. The method according to claim 42, further comprising calculating the number or concentration of viable cells of the first population present in the sample from the amount of liberated ATP that is determined.

56. The method according to claim 40, wherein the method is a sequencing-by-synthesis procedure, the contaminating nucleotides comprise excess dNTPs or analogues thereof present after a completed sequencing cycle, and the analysis performed on the sample is a sequence readout.

57. The method according to claim 40, wherein the method further comprises:
performing a pyrosequencing reaction comprising adding a nucleoside triphosphate and releasing a pyrophosphate;
converting the released pyrophosphate into ATP via an enzymatic reaction;
determining the amount of ATP formed;
degrading unincorporated nucleoside triphosphate and ATP with an apyrase enzyme, wherein the apyrase enzyme is a Shigella flexneri apyrase;
repeating the performing, converting, determining, and degrading steps at least once.

58. The method according to claim 41, wherein determining the amount of formed ATP is performed with a bioluminescent assay.

59. The method according to claim 58, wherein the bioluminescent assay utilizes luciferin and luciferase.

60. The method according to claim 59, wherein the luciferase is provided in a composition comprising an apyrase inhibitor comprising ortho-vanadate.

61. The method according to claim 40, wherein the Shigella flexneri apyrase comprises an amino-acid sequence having at least 80% sequence identity to SEQ ID NO: 10.

62. The method according to claim 40, wherein the Shigella flexneri apyrase comprises an amino-acid sequence according to SEQ ID NO: 4.

63. The method according to claim 40, wherein Shigella flexneri apyrase is in a buffer having a pH in the range of 6-9, and an ionic strength of at least 300 mM.

64. A Shigella flexneri apyrase comprising an amino-acid sequence having at least 97% sequence identity to SEQ ID NO: 4 and having apyrase activity.

65. The Shigella flexneri apyrase according to claim 64, comprising an amino-acid sequence according to SEQ ID NO: 4.

66. A composition comprising a Shigella flexneri apyrase in a buffer having a pH in the range of 6-9, and an ionic strength of at least 300 mM.

67. The composition according to claim 66, wherein the ionic strength is 300-1000 mM.


SEQUENCE LISTING (10 sequences)

SEQ ID NO: 1 - DNA, 1365 bp, Solanum tuberosum
atgttgaaccaaaatagtcattttgttttcacaattttggcaatatttttggttttgcccctaagtttattatccaaaaatgtggatgctcaaattccattaagaagacatttattaagtcatgaatcagaacattatgcagtaatatttgatgctggaagtactggaagtagagttcatgtttttcgatttgatgaaaaattaggacttcttcctattggcaacaatattgagtattttatggcgacagagccaggtttaagttcatatgcagaagatccaaaggctgcagccaattcacttgagccacttttagatggagctgaaggagttgttcctcaagaattgcaatctgaaacacctttagaacttggggcaacagcaggtcttaggatgttaaaaggggatgcagctgaaaaaattctacaagcagtgagaaatttagtgaagaatcaaagcactttccatagcaaagatcaatgggtcactattcttgatggaactcaagaaggctcttatatgtgggctgcaataaattatttattgggaaatttgggcaaagattataaaagtacaacagcaacaattgatcttggtggtggttcagtccaaatggcatatgctatatcaaatgaacaatttgcaaaagctcctaataatgaggatggagaaccttatgtacaacaaaaacatcttatgtcaaaagattataatctctatgtacatagttatttaaactatgggcaattagcaggtcgagctgagattttcaaggcttcaagaaatgaaagtaatccttgtgctctggaaggatatgatgggtattactcatatggaggagtggactacaaagtaaaagcaccaaagaaaggtagtagttggaagagatgcaggaggttaactagacatgcacttaaaataaatgcaaaatgcaagattgaagaatgcaccttcaatggagtgtggaatggtggtggtggtgatggacaaaaaaatattcatgcttcatcatttttttatgatattggtgctcaggttggcattgttgacaccaaatttccatctgctctagcaaagccaattcaatacttaaatgcagctaaagttgcttgccaaacaaatgtggcagatataaaatccatattcccaaaaactcaagatagaaatatcccatacttatgtatggacttgatatatgagtacactttgcttgttgatggatttggactaaatccacacaaagaaataacagtgatacatgatgtgcaatacaaaaactatctagttggagcagcatggccattgggatgtgccattgacttggtttcttcaactacaaacaaaattagagttgcatcatcttaa

SEQ ID NO: 2 - DNA, 1134 bp, Shigella flexneri
tcacaaatcatcataatcaagagacaaaacgatacgaaaaaataagataaaaaacatcgttcttttaccacattattttccgtgaatatgaaaaataatgttattactttaatataagactattttttgtttttccatcactctgttcaaatttttccgcatgacttgtgttttttgtaatacagctcgttttttacagctgaccaaaatcatcaattaattatgctaaggaaataaattatgaaaaccaaaaactttcttcttttttgtattgctacaaatatgatttttatcccctcagcaaatgctctgaaggcagaaggttttctcactcaacaaacttcaccagacagtttgtcaatacttccgccgcctccggcagagaattcagtagtatttcaggctgacaaagctcattatgaattcggccgctcgctccgggatgctaatcgtgtacgtctcgctagcgaagatgcatactacgagaattttggtcttgcattttcagatgcttatggcatggatatttcaagggaaaataccccaatcttatatcagttgttaacacaagtactacaggatagccatgattacgccgtgcgtaacgccaaagaatattataaaagagttcgtccattcgttatttataaagacgcaacctgtacacctgataaagatgagaaaatggctatcactggctcttatccctctggtcatgcatcctttggttgggcagtagcactgatacttgcggagattaatcctcaacgtaaagcggaaatacttcgacgtggatatgagtttggagaaagtcgggtcatctgcggtgcgcattggcaaagcgatgtagaggctgggcgtttaatgggagcatcggttgttgcagtacttcataatacacctgaatttaccaaaagccttagcgaagccaaaaaagagtttgaagaattaaatactcctaccaatgaactgaccccataaagctggacagcctgtatcaggctatggagggcccatagacaaatctaccctatatgagcataggaggagtctatgggcacaccacgttttacccctgaatttaagggattactggaaaggctgggacatatcctccggcagaagcagaaaaag

SEQ ID NO: 3 - DNA, 780 bp, Shigella flexneri
gatatacatatgaaaaccaaaaactttcttcttttttgtattgctacaaatatgatttttatcccctcagcaaatgctctgaaggcagaaggttttctcactcaacaaacttcaccagacagtttgtcaatacttccgccgcctccggcagagaattcagtagtatttcaggctgacaaagctcattatgaattcggccgctcgctccgggatgctaatcgtgtacgtctcgctagcgaagatgcatactacgagaattttggtcttgcattttcagatgcttatggcatggatatttcaagggaaaataccccaatcttatatcagttgttaacacaagtactacaggatagccatgattacgccgtgcgtaacgccaaagaatattataaaagagttcgtccattcgttatttataaagacgcaacctgtacacctgataaagatgagaaaatggctatcactggctcttatccctctggtcatgcatcctttggttgggcagtagcactgatacttgcggagattaatcctcaacgtaaagcggaaatacttcgacgtggatatgagtttggagaaagtcgggtcatctgcggtgcgcattggcaaagcgatgtagaggctgggcgtttaatgggagcatcggttgttgcagtacttcataatacacctgaatttaccaaaagccttagcgaagccaaaaaagagtttgaagaattaaatactcctaccaatgaactgaccccacatcatcaccatcaccactaacgtacgatgttc

SEQ ID NO: 4 - protein, 259 aa, Shigella flexneri
DIHMKTKNFLLFCIATNMIFIPSANALKAEGFLTQQTSPDSLSILPPPPAENSVVFQADKAHYEFGRSLRDANRVRLASEDAYYENFGLAFSDAYGMDISRENTPILYQLLTQVLQDSHDYAVRNAKEYYKRVRPFVIYKDATCTPDKDEKMAITGSYPSGHASFGWAVALILAEINPQRKAEILRRGYEFGESRVICGAHWQSDVEAGRLMGASVVAVLHNTPEFTKSLSEAKKEFEELNTPTNELTPHHHHHHRTMF

SEQ ID NO: 5 - DNA, 28 bp, Artificial
  Primer
cgcggatccctgaaggcagaaggttttc

SEQ ID NO: 6 - DNA, 27 bp, Artificial
  Primer
cccaagcttttatggggtcagttcatt

SEQ ID NO: 7 - DNA, 53 bp, Artificial
  Primer
gaacatcgtacgttagtggtgatggtgatgatgtggggtcagttcattggtag

SEQ ID NO: 8 - DNA, 28 bp, Artificial
  Primer
gatatacatatgaaaaccaaaaactttc

SEQ ID NO: 9 - protein, 454 aa, Solanum tuberosum
MLNQNSHFIFIILAIFLVLPLSLLSKNVNAQIPLRRHLLSHESEHYAVIFDAGSTGSRVHVFRFDEKLGLLPIGNNIEYFMATEPGLSSYAEDPKAAANSLEPLLDGAEGVVPQELQSETPLELGATAGLRMLKGDAAEKILQAVRNLVKNQSTFHSKDQWVTILDGTQEGSYMWAAINYLLGNLGKDYKSTTATIDLGGGSVQMAYAISNEQFAKAPQNEDGEPYVQQKHLMSKDYNLYVHSYLNYGQLAGRAEIFKASRNESNPCALEGCDGYYSYGGVDYKVKAPKKGSSWKRCRRLTRHALKINAKCNIEECTFNGVWNGGGGDGQKNIHASSFFYDIGAQVGIVDTKFPSALAKPIQYLNAAKVACQTNVADIKSIFPKTQDRNIPYLCMDLIYEYTLLVDGFGLNPHKEITVIHDVQYKNYLVGAAWPLGCAIDLVSSTTNKIRVASS

SEQ ID NO: 10 - protein, 246 aa, Shigella flexneri
MKTKNFLLFCIATNMIFIPSANALKAEGFLTQQTSPDSLSILPPPPAENSVVFQADKAHYEFGRSLRDANRVRLASEDAYYENFGLAFSDAYGMDISRENTPILYQLLTQVLQDSHDYAVRNAKEYYKRVRPFVIYKDATCTPDKDEKMAITGSYPSGHASFGWAVALILAEINPQRKAEILRRGYEFGESRVICGAHWQSDVEAGRLMGASVVAVLHNTPEFTKSLSEAKKEFEELNTPTNELTP